(19) United States
(12) Patent Application Publication
(10) Pub. No.: US 2018/0362343 A1
(43) Pub. Date: Dec. 20, 2018

(54) METHOD FOR THE PREPARATION OF BIS(FLUOROSULFONYL)-IMIDE AND OF ITS SALTS

(71) Applicant: LONZA LTD, Visp (CH)

(72) Inventors: Anna-Christina HORMES, Visp (CH); Andreas KLEIN, Brig-Glis (CH); Christian SCHNIDER, Visp (CH); Thomas GRUETZNER, Thun (CH); Michael BERSIER, Ausserberg (CH); Philipp STUDER, Visp (CH); Stefan TILLE, Naters (CH); Desirée LORENZ, Törbel (CH); Hilke-Marie LORENZ, Thun (CH); Hans HASSE, Kaiserslautern (DE); Erik VON HARBOU, Neustadt / Weinstraße (DE); Johannes NEUHAUS, Kaiserslautern (DE)

(21) Appl. No.: 15/775,687

(22) PCT Filed: Oct. 26, 2016
(86) PCT No.: PCT/EP2016/075836

§ 371 Date: May 11, 2018

Related U.S. Application Data
(60) Provisional application No. 62/254,860, filed on Nov. 13, 2015.
(60) Provisional application No. 62/290,523, filed on Feb. 3, 2016.
(60) Provisional application No. 62/308,313, filed on Mar. 15, 2016.

(30) Foreign Application Priority Data
Nov. 13, 2015 (EP) 15194513.6
Nov. 18, 2015 (EP) 15195231.4
Feb. 3, 2016 (EP) 16153983.8
Mar. 15, 2016 (EP) 16160245.3
Mar. 15, 2016 (EP) 16160248.7
Mar. 30, 2016 (EP) 16163043.9
Mar. 30, 2016 (EP) 16163045.4
Apr. 5, 2016 (EP) 16163835.8
Apr. 6, 2016 (EP) 16164023.0
Apr. 7, 2016 (EP) 16164146.9
Apr. 7, 2016 (EP) 16164148.5
Apr. 8, 2016 (EP) 16164371.3
Apr. 8, 2016 (EP) 16164373.9
Apr. 11, 2016 (EP) 16164593.2
Apr. 11, 2016 (EP) 16164595.7
Apr. 29, 2016 (EP) 16167626.7
Apr. 29, 2016 (EP) 16167634.1
Jul. 20, 2016 (EP) 16180254.1
Jul. 20, 2016 (EP) 16180255.8
Jul. 20, 2016 (EP) 16180274.9
Sep. 13, 2016 (EP) 16188590.0
Sep. 13, 2016 (EP) 16188591.8
Sep. 19, 2016 (EP) 16189382.1
Sep. 19, 2016 (EP) 16189383.9
Sep. 19, 2016 (EP) 16189474.6
Sep. 19, 2016 (EP) 16189478.7
Oct. 11, 2016 (EP) 16193206.6
Oct. 25, 2016 (EP) 16195489.6

(51) Int. Cl. C01B 21/086; C01B 21/093; C07C 303/40

(52) U.S. Cl. CPC C01B 21/086; C07C 303/40; C01B 21/093

(57) ABSTRACT

The invention relates to a method for the preparation of bis(fluorosulfonyl)-imide, the method starts from bis(chlorosulfonyl)-imide or its respective derivatives, which is reacted with HF in the presence of chlorosulfonyl isocyanate, and uses a certain extraction step for extraction of bis(fluorosulfonyl)-imide from an aqueous solution; the invention is also useful for the preparation of certain salts of bis(fluorosulfonyl)-imide and its derivatives.

### CROSS-REFERENCE TO RELATED APPLICATIONS

This application is a U.S. National Stage application of PCT/EP2016/075836 filed 26 Oct. 2016, which claims priority to U.S. Provisional Patent Application No. 62/254,860 filed 13 Nov. 2015, European Patent Application No. 15194513.6 filed 13 Nov. 2015, European Patent Application No. 15195231.4 filed 18 Nov. 2015, U.S. Provisional Patent Application No. 62/290,523 filed 3 Feb. 2016, European Patent Application No. 16153983.8 filed 3 February 2016, European Patent Application No. 16160245.3 filed 15 Mar. 2016, U.S. Provisional Patent Application No. 62/308,313 filed 15 Mar. 2016, European Patent Application No. 16160248.7 filed 15 Mar. 2016, European Patent Application No. 16163043.9 filed 30 Mar. 2016, European Patent Application No. 16163045.4 filed 30 Mar. 2016, European Patent Application No. 16163835.8 filed 5 Apr. 2016, European Patent Application No. 16164023.0 filed 6 Apr. 2016, European Patent Application No. 16164146.9 filed 7 Apr. 2016, European Patent Application No. 16164148.5 filed 7 Apr. 2016, European Patent Application No. 16164371.3 filed 8 Apr. 2016, European Patent Application No. 16164373.9 filed 8 Apr. 2016, European Patent Application No. 16164593.2 filed 11 Apr. 2016, European Patent Application No. 16164595.7 filed 11 Apr. 2016, European Patent Application No. 16167626.7 filed 29 Apr. 2016, European Patent Application No. 16167634.1 filed 29 Apr. 2016, European Patent Application No. 16180254.1 filed 20 Jul. 2016, European Patent Application No. 16180255.8 filed 20 Jul. 2016, European Patent Application No. 16180274.9 filed 20 Jul. 2016, European Patent Application No. 16188590.0 filed 13 Sep. 2016, European Patent Application No. 16188591.8 filed 13 Sep. 2016, European Patent Application No. 16189382.1 filed 19 Sep. 2016, European Patent Application No. 16189474.6 filed 19 Sep. 2016, European Patent Application No. 16189383.9 filed 19 Sep. 2016, European Patent Application No. 16189478.7 filed 19 Sep. 2016, European Patent Application No. 16193206.6 filed 11 Oct. 2016, European Patent Application No. 16195489.6 filed 25 Oct. 2016, the entire disclosures of which are hereby incorporated by reference in their entireties.

The invention relates to a method for the preparation of bis(fluorosulfonyl)-imide, the method starts from bis(chlorosulfonyl)-imide or its respective derivatives, which is reacted with HF in the presence of chlorosulfonyl isocyanate, and uses a certain extraction step for extraction of bis(fluorosulfonyl)-imide from an aqueous solution; the invention is also useful for the preparation of certain salts of bis(fluorosulfonyl)-imide and its derivatives.

### BACKGROUND OF THE INVENTION

In the following text, the following meanings are used, if not otherwise stated:
ACN acetonitrile;
ClSI bis(chlorosulfonyl)-imide, that is compound of formula (2);

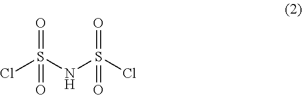

CSI chlorosulfonyl isocyanate, that is compound of formula (3);

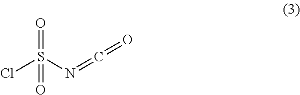

CSOS, CSA chlorosulfonic acid;
DCB dichlorobenzene, if not otherwise stated it is 1,2-dichlorobenzene;
DCE dichloroethane, if not otherwise stated it is 1,2-dichloroethane;
DCM dichloromethane;
DFACl difluoro acetic acid chloride;
DFAF difluoro acetic acid fluoride;
halide stands for fluoride, chloride, bromide or iodide, preferably fluoride, chloride or bromide, more preferably fluoride or chloride, even more preferably chloride;
halogen F, Cl, Br or I, preferably F, Cl or Br, more preferably F or Cl.
HFSI bis(fluorosulfonyl)-imide, that is compound of formula (1);

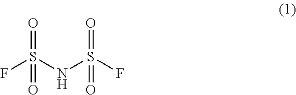

LiFSI Lithium bis(fluorosulfonyl)-imide, that is compound of formula (5);

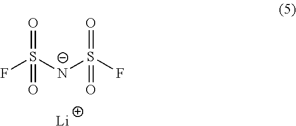

Mp melting point;
MTBE methyl-tert butyl ether;
TEA triethylamine;
THF tetrahydrofuran;
MeTHF 2-methyl tetrahydrofuran;
VN valeronitrile;
wt %, % by weight percent by weight.

For the purpose of this invention, distillation and evaporation means essentially the same, that is a vaporizing of a volatile compound; this is done preferably to remove said volatile compound from a mixture. The difference between distillation and evaporation lies primarily in the type of devices used for said vaporizing. Therefore the two terms distillation and evaporation are used interchangeably herein, if not stated otherwise.

Salts of bis(fluorosulfonyl)-imide, as for example LiFSI, are used for the production of electrolytes in electrochemical devices, examples are lithium ion batteries. HFSI is an intermediate used for the production salts of bis(fluorosulfonyl)-imide.

US 2013/0331609 A1 discloses a method for producing a metal salt of fluorosulfonyl imide in two steps, in a first step di(chlorosulfonyl)imide is reacted with the flourinating agent NH₄F providing the ammonium di(fluorosulfonyl)imide, in a second step the ammonium di(fluorosulfonyl)imide is converted with LiOH to lithium di(flurosulfonyl)imide. The first step is done in the presence of a solvent, in the example acetonitrile is used and the reaction was performed under reflux conditions at 80 to 84° C. for 4 hours.

WO 2009/123328 A1 discloses a method for preparation of metal salts of symmetrical and asymmetrical fluorosulfonylimide in a solvent by a reaction of a respective symmetrical or asymmetrical chlorosulfonylimide with a fluoride compound containing at least one element selected from the group consisting of elements of Group 11 to Group 15 and Period 4 to Period 6 (excluding arsenic and antimony), these metal salts are then converted in a second step to salts of various amines and symmetrical and asymmetrical fluorosulfonylimide in a cation exchange reaction.

US 2015/0246812 A1 discloses a method for the preparation of symmetrical and asymmetrical flourosulfonylimides from symmetrical and asymmetrical chlorosulfonylimides, wherein the reaction is done in an organic solvent.

WO 2015/012897 A1 discloses a method for producing FSI from ClSI using HF, wherein the HCl that is produced by the reaction is selectively removed during the reaction to produce HFSI in at least 80% yield. The reaction takes place at ambient (e.g. atmospheric) pressure. Reaction times are much longer than 3 hours. Both requirements, the rather long reaction times and the requirement for separating HCl from the reaction mixture during the reaction, require a special continuous stirred-tank reactor (“CSTR”) set-up with a device for the required separation of HCl during the reaction when carrying out the reaction in a continuous way. To do the reaction in a simple continuously working tube shaped reactor creates problems.

Also disclosed is the exchange of Br and I instead of Cl against F, that is the conversion of hydrogen bis(halosulfonyl)imide (HXSI) with hydrogen fluoride for producing hydrogen bis(fluorosulfonyl)imide (HFSI), where each X is independently a nonfluoro-halide, such as CI, Br, or I.

WO 2015/004220 A1 discloses a method for the preparation of imidodisulfuryl compounds in a continuous reaction at elevated temperatures.

U.S. Pat. No. 7,919,629 B2 discloses in Example 10 the reaction of distilled ClSI, which was obtained by distillation under vacuum, with HF and reports i.a. 55% yield for the example with 2 h at 130° C. Reproduction of this example 10 in Comparative Example (i) and determination of the CSI content revealed a residual content of 0.3 wt-% of CSI in the starting material ClSI which was obtained by said distillation under vacuum. Example 8 according to present invention shows a considerable higher yield of 82%.

Rolf Appel et al, Chemische Berichte, 1962, 95, 1753-1755, discloses on page 1755 the preparation of ClSI from CSI and CSOS. The final product is obtained from distillation of the crude product under vacuum. According to Comparative Example (i) a residual content of 0.3 wt-% can be assumed in the ClSI after distillation under vacuum.

EP 0 055 899 A2 discloses in example 1 the preparation of ClSI from CSI and CSOS. The final product is obtained from distillation of the crude product under vacuum, and this distilled product is then used for further reactions. According to Comparative Example (i) a residual content of 0.3 wt-% can be assumed in the ClSI after distillation under vacuum.

EP 2 662 332 A discloses in example 4 a method for preparation of ammonium di(fluorosulfonyl)imide by reacting di(chlorosulfonyl)imide with NH₄F in ethyl acetate.

EP 2 660 196 A discloses a method for preparation of ammonium di(fluorosulfonyl)imide by reacting di(chlorosulfonyl)imide and NH₄F (HF)ₚ. According to [0030] the reaction is done in an organic solvent, that is preferably dewatered prior to use, or it is done in the absence of a solvent. Example 1 uses acetonitrile as solvent.

EP 2 674 395 A discloses in [102] a process for producing the ammonium salt of di(fluorosulfonyl)-imide, wherein anhydrous hydrogen fluoride in acetonitrile is reacted with ammonium di(chlorosulfonyl)imide. Addition of ethylacetat and water follows, the organic phase was separated and the water phase was extracted 3 times with ethyl acetate. The organic phases obtained in the extraction operations were combined, and the combined organic phase was washed with water, and ammonium di(fluorosulfonyl)-imide was isolated from the organic phase. The ammonium di(fluorosulfonyl)-imide stays in the organic phase all the time.

EP 2 505 551 A1 discloses in Experimental Example 1 a fluorination reaction between di(chlorosulfonyl) imide and ZnF₂. The reaction is done in butyl acetate. The reaction solution is added after the reaction to ammonia water. Two phases are formed, the water phase is removed, the desired fluorosulfonylimide is in the organic layer in form of the ammonium salt of di(chlorosulfonyl) imide.

EP 2 578 533 A1 discloses in Experimental Example 1-1 a fluorination reaction between di(chlorosulfonyl) imide and ZnF₂. The reaction is done in butyl acetate. The reaction solution is added after the reaction to ammonia water. Two phases are formed, the water phase is removed, the desired fluorosulfonylimide is in the organic layer in form of the ammonium salt of di(chlorosulfonyl) imide.

There was a need for a method for preparation of salts of bis(fluorosulfonyl)-imide starting from ClSI that does not require mandatorily a solvent in the fluorination reaction, that does not require mandatorily metal salts in the fluorination reaction, and that has few steps, that produces salts of bis(fluorosulfonyl)-imide in high yields and where the fluorination reaction both batch wise and in a continuous manner in a continuous reactor, and also in a continuous tube shape reactor. The method should allow for the fluorination with HF, and should not require the use of sources of F in other forms than HF, such as NH₄F or ZnF₂.

The method should allow for purification of HFSI that can be easily incorporated into the method for preparation of HFSI, which allows for example removal of water soluble impurities.

Furthermore the method should allow for the preparation and purification of HFSI without mandatorily requiring the formation of a salt of HFSI such as an ammonium salt.

Furthermore the method should allow for subsequent preparation of salts of HFSI, such as LiFSI. The method should allow the preparation of said salts in high yields. It should allow to be done both batch wise and in a continuous manner in a continuous reactor, and also in a continuous tube shape reactor.

The method should not require the separation of HCl during the fluorination reaction for enhancement of the yield, as it is disclosed in WO 2015/012897 A1 and should allow to carry out the fluorination reaction in relatively short reaction times.

It was found that it is possible to purify HFSI from water and not from an organic solvent, and to extract HFSI from water with an organic solvent, without mandatorily requiring the use of ammonia or the formation of the ammonium salt of HFSI.

This purification can be used for the preparation of HFSI and in the preparation of salts of HFSI.

The method of present invention for purification or preparation of HFSI and for preparation of salts of bis(fluorosulfonyl)-imide can start from ClSI and can proceed via HFSI as intermediate, it does not require a solvent in the fluorination reaction, it does not require metal salts in the fluorination reaction, it uses F in form of HF, it has few steps, it produces HFSI in the fluorination reaction in high yields in spite of the poor solubility and miscibility of HF in ClSI and vice versa, and the fluorination reaction can be done both batch wise or in a continuous manner and also in a continuous tube shape reactor, and the method is distinguished by short reaction times especially in the fluorination reaction.

The method does not require the separation of HCl during the fluorination reaction, which is formed by the fluorination reaction, and still provides the intermediate HFSI in good yields. This was unexpected in view of the disclosure of WO 2015/012897 A1. Furthermore it was unexpected that the use of ClSI containing CSI in the reaction with HF provides for significantly higher yield than the use of ClSI which was obtained by distillation as disclosed in U.S. Pat. No. 7,919,629 B2. This is exemplified herein with Comparative Example (i) versus Example 8.

None of the prior art discloses the use of ClSI for the preparation of HFSI, where this ClSI contains deliberately CSI, instead the various preparation examples in the prior art always end with a distillation of the ClSI, which is clearly meant for purification of ClSI for further use, and the residual content of CSI after such a distillation was determined to be only 0.3 wt-%. There is also no motivation or hint in the prior art to carry out the preparation of HFSI in the presence of CSI, and there is no hint in the prior art that the presence of CSI might increase the yield of HFSI. Especially for the use of LiFSI in batteries, the purity of LiFSI is a critical issue and various patent applications take this requirement into consideration by claiming LiFSI in high purities. Also for this reason the skilled person would not consider starting with a ClSI which is not purified, for example by distillation, and that would therefore contain major amounts of impurities, these impurities leading to by products in the reactions to HFSI and in the reactions to the salts of HFSI. These by products would need to be separated in order to attain the high purity profile of LiFSI that is required in batteries.

The fluorination reaction can be done with relatively short reaction times compared to the disclosure in the prior art, which allows to do the fluorination reaction not only batch wise, but also in continuous manner, also in a continuous tube shape reactor.

### SUMMARY OF THE INVENTION

Subject of the invention is a method for preparation of compound of formula (I)

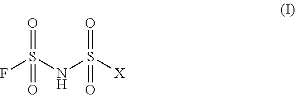

the method comprises a step STEP1, a step STEPMIX and a step STEPEXTR;
STEP1 comprises a reaction REAC1-1;
in REAC1-1 a compound of formula (II) is reacted with HF

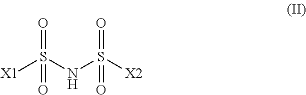

at a temperature TEMP1-1, TEMP1-1 is at least 80° C.;
wherein at the beginning of REAC1-1 compound of formula (III) is present in the reaction mixture;

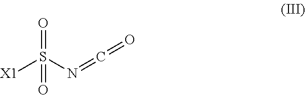

the amount of compound of formula (III), that is present in the reaction mixture at the beginning of REAC1-1, is at least 0.5%, the % are % by weight and are based on the weight of the reaction mixture at the beginning of REAC1-1 excluding from said weight of the reaction mixture the weight of the HF;
X is identical with X1 or with X2;
X1 and X2 are identical or different and independently from each other selected from the group consisting of F, Cl, Br, I, RESF, and tolyl;
with the proviso that at least one of the residues X1 and X2 is Cl, Br, or I;
RESF is fluorinated C₁₋₉ alkyl, which is unsubstituted or substituted by a substituent OCF₃;
in STEPMIX compound of formula (I) is mixed with water by a mixing MIX, MIX provides a mixture MIXWAT, MIXWAT is the mixture of compound of formula (I) with water,
in STEPEXTR compound of formula (I) is extracted from MIXWAT by an extraction EXTR, EXTR is the extraction of compound of formula (I) from MIXWAT with an organic solvent SOLVORG, SOLVORG is an organic solvent that forms a biphasic system with water;
EXTR provides compound of formula (I) in form of a solution SOLCOMP1, SOLCOMP1 is the solution of compound of formula (I) in SOLVORG.

### DETAILED DESCRIPTION OF THE INVENTION

“Fluorinated alkyl” means, that at least one H is exchanged for F.

The reaction product of REAC1-1 is compound of formula (I).

Preferably,
RESF is fluorinated C₁₋₆ alkyl, which is unsubstituted or substituted by a substituent OCF₃; more preferably,
RESF is fluorinated C₁₋₄ alkyl, which is unsubstituted or substituted by a substituent OCF₃; even more preferably,
RESF is fluorinated C₁₋₂ alkyl, which is unsubstituted or substituted by a substituent OCF₃.

Especially, any RESF herein is a perfluoroalkyl.

Specific embodiments of RESF are for example CF₃, CHF₂, CH₂F, C₂F₅, C₂HF₄, C₂H₂F₃, C₂H₃F₂, C₂H₄F, C₃F₇, C₃HF₆, C₃H₂F₅, C₃H₄F₃, C₃H₆F, C₄F₉, C₄H₂F₇, C₄H₄F₅, C₄H₈F, C₅F₁₁, C₅H₁₀F, C₃F₆OCF₃, C₂F₄OCF₃, C₂H₂F₂OCF₃, CF₂OCF₃, C₆F₁₃, C₆H₁₂F, C₇F₁₅, C₈F₁₇ and C₉F₁₉;
preferably CF₃, CHF₂, CH₂F, fluoroethyl, difluoroethyl, 2,2,2-trifluoroethyl, pentafluoroethyl, 3,3,3-trifluoropropyl, a perfluoro-n-propyl, fluoropropyl, perfluoroisopropyl, fluorobutyl, 3,3,4,4,4-pentafluorobutyl, perfluoro-n-butyl, perfluoroisobutyl, perfluoro-t-butyl, perfluoro-sec-butyl, fluoropentyl, perfluoropentyl, perfluoroisopentyl, perfluoro-t-pentyl, fluorohexyl, perfluoro-n-hexyl and perfluoroisohexyl;
more preferably, trifluoromethyl, pentafluoroethyl and perfluoro-n-propyl;
even more preferably, trifluoromethyl and pentafluoroethyl.

Preferably,
X1 and X2 are identical or different and independently from each other selected from the group consisting of F, Cl, Br, I, RESF, RESF being preferably C₁₋₆ perfluoroalkyl, and tolyl;
more preferably,
X1 and X2 are identical or different and independently from each other selected from the group consisting of F, Cl, Br, RESF, RESF being preferably C₁₋₆ perfluoroalkyl, and tolyl;
even more preferably,
X1 and X2 are identical or different and independently from each other selected from the group consisting of F, Cl, and RESF, RESF being preferably C₁₋₄ perfluoroalkyl;
especially,
X1 and X2 are identical or different and independently from each other selected from the group consisting of Cl, and RESF, RESF being preferably C₁₋₂ perfluoroalkyl;
more especially,
X1 and X2 are identical or different and independently from each Cl or CF₃.

Preferably,
X is selected from the group consisting of F, Cl, Br, I, RESF, and tolyl.

More preferably,
X is selected from the group consisting of F, Cl, Br, I, RESF, RESF being preferably C₁₋₆ perfluoroalkyl, and tolyl.

Even more preferably,
X is selected from the group consisting of F, Cl, Br, RESF, RESF being preferably C₁₋₆ perfluoroalkyl, and tolyl.

Especially,
X is selected from the group consisting of F, Cl, and RESF, RESF being preferably C₁₋₄ perfluoroalkyl.

More especially,
X is selected from the group consisting of Cl, and RESF, RESF being preferably C₁₋₂ perfluoroalkyl.

Even more especially,
X is Cl or CF₃.

Specific embodiments of compound of formula (I) are compound of formula (1) and compound of formula (1-CF₃).

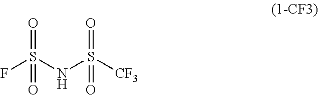 [SMILES: O=S(=O)(F)NS(=O)(=O)C(F)(F)F]

Specific embodiments of compound of formula (II) are compound of formula (2) and compound of formula (2-CF₃).

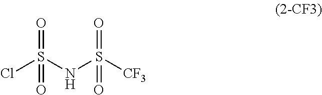 [SMILES: O=S(=O)(Cl)NS(=O)(=O)C(F)(F)F]

Specific embodiment of compound of formula (III) is compound of formula (3).

Compound of formula (III) can also react during REAC1-1, and if this happens then compound of formula (III) is not necessarily present in REAC1-1 from the beginning to the end. So the terminology “at the beginning of REAC1-1 compound of formula (III) is present in the reaction mixture” comprises also the case that REAC1-1 is started in the presence of compound of formula (III), or that compound of formula (III) is present in the reaction mixture at the beginning of REAC1-1, and it comprises also the case where compound of formula (III) is present in REAC1-1 or during REAC1-1, and it comprises also the case that the amount of compound of formula (III) decreases during REAC1-1.

Preferably, the amount of compound of formula (III), that is present in the reaction mixture at the beginning of REAC1-1, is at least 0.5%, more preferably at least 0.75%, even more preferably at least 1%, especially at least 2%, more especially at least 3%, even more especially at least 4%, the % are % by weight and are based on the weight of the reaction mixture at the beginning of REAC1-1 excluding from said weight of the reaction mixture the weight of the HF.

Preferably, not more than 50%, more preferably not more than 25%, even more preferably not more than 15%, especially not more than 12.5%, more especially not more than 10%, of compound of formula (III) is present in the reaction mixture at the beginning of REAC1-1, the % are % by weight and are based on the weight of the reaction mixture at the beginning of REAC1-1 excluding from said weight of the reaction mixture the weight of the HF.

Any of the lower limits can be combined with any of the upper limits of the amount of compound of formula (III) that is present at the beginning of REAC1-1.

Preferably, REAC1-1 is done in a continuous way.

STEP1 can comprise a purification PUR1, in PUR1 compound of formula (I) is purified by extraction, distillation, evaporation, membrane assisted separation, or a combination thereof;

preferably by distillation or evaporation.

PUR1 is done after REAC1-1.

Membrane assisted separation is preferably membrane assisted pervaporation or vapor permeation, or membrane assisted filtration.

Preferably, distillation or evaporation is done by using a film evaporator, wiped film evaporator, falling film evaporation, rectification, flash distillation, short path distillation, or a combination thereof;
more preferably distillation or evaporation is done by using a falling film evaporation, rectification, wiped film evaporator, or a combination thereof;
even more preferably, distillation or evaporation is done by using a falling film evaporation combined with a rectification or wiped film evaporator.

Preferably, PUR1 is done continuously.

Further subject of the invention is a method for purification of compound of formula (I), wherein the method comprises the step STEPMIX and the step STEPEXTR.

Preferably, MIXWAT has a content of from 0.5 to 50%, more preferably of from 0.5 to 35%, even more preferably of from 0.5 to 20%, especially of from 1 to 10%, more especially of from 2 to 8%, of compound of formula (I), the % are % by weight and are based on the combined amount of water and compound of formula (I), preferably based on the weight of MIXWAT.

Therefore compound of formula (I) and water are mixed in such a ratio so as to obtain said content of compound of formula (I) in water.

MIX can be done by charging water to compound of formula (I) or by charging compound of formula (I) to water.

MIX can be done batchwise or in a continuous way, preferably MIX is done continuously.

Preferably, MIX is done preferably by using a mixer, preferably a static mixer. Such mixers are known to the skilled person.

Preferably, MIX is done in the absence of an organic solvent.

Preferably, MIX is done in the absence of a solvent other than water.

Preferably, MIX is done in the absence of an organic base containing nitrogen.

Preferably, MIX is done in the absence of a salt of an organic base containing nitrogen.

Preferably, MIX is done in the absence of an organic base.

Preferably, MIX is done in the absence of a salt of an organic base.

Preferably, MIX is done in the absence of a base.

Preferably, MIX is done in the absence of a salt of a base.

Preferably, MIX is done at a temperature of from −5 to 50° C., more preferably of from 0 to 40° C.

Preferably, MIX is done at ambient pressure. It is possible to do MIX at elevated pressure, preferably at a pressure of from ambient pressure to 10 bar, more preferably of from ambient pressure to 5 bar, even more preferably of from ambient pressure to 2.5 bar.

Preferably, the mixing time TIMEMIX of MIX is from 1 min to 2 h, more preferably from 2 min to 1.5 h, even more preferably 5 min to 1 h, especially from 5 min to 30 min.

In another preferred embodiment, TIMEMIX is from 5 min to 10 h.

Preferably, SOLVORG is selected from the group consisting of carbonate-based solvent, aliphatic ether-based solvent, ester-based solvent, amide-based solvent, nitro-based solvent, sulfur-based solvent, nitrile-based solvent, keton based solvent, and mixtures thereof.

Preferably, SOLVORG is selected from the group consisting of compound of formula (SOLVORG-I), compound of formula (SOLVORG-II), compound of formula (SOLVORG-III), compound of formula (SOLVORG-IV), compound of formula (SOLVORG-V), compound of formula (SOLVORG-VI), compound of formula (SOLVORG-VII), compound of formula (SOLVORG-VIII), compound of formula (SOLVORG-X), compound of formula (SOLVORG-XI), and compound of formula (SOLVORG-XII);

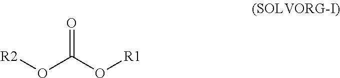

R1 and R2 are identical or different and are C₁₋₄ alkyl; or
R1 and R2 form together a CH₂CH₂ chain which connects the two oxygen atoms and thereby forms a 5 membered ring, the CH₂CH₂ chain being unsubstituted or substituted by 1 or 2 C₁₋₄ alkyl residues;
   preferably,
R1 and R2 are identical or different and are C₁₋₂ alkyl; or
R1 and R2 form together a CH₂CH₂ chain which connects the two oxygen atoms and thereby forms a 5 membered ring, the CH₂CH₂ chain being unsubstituted or substituted by C₁₋₂ alkyl;

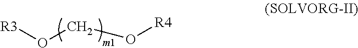

R3 and R4 are identical or different and independently from each other C₁₋₄ alkyl, and
m1 is 1, 2, 3 or 4;
   preferably,
R3 and R4 are identical or different and independently from each other C₁₋₂ alkyl, and
m1 is 1 or 2;

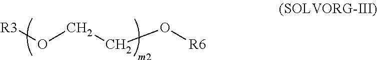

R5 and R6 are identical or different and independently from each other C₁₋₄ alkyl, and
m2 is 2, 3 or 4;
   preferably,
R5 and R6 are identical or different and independently from each other C₁₋₂ alkyl, and
m2 is 2 or 3;

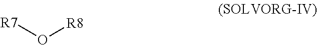

R7 and R8 are identical or different and independently from each other C₁₋₆ alkyl or C₅₋₆ cyclo alkyl;
   preferably,
R7 and R8 are identical or different and independently from each other C₂₋₅ alkyl or C₅ cyclo alkyl,

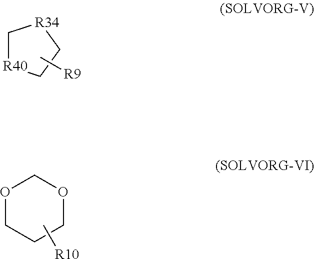

R9 and R10 are identical or different and independently from each other H or C₁₋₄ alkyl,
R34 is O or SO₂, and
R40 is CH₂ or O;
   preferably,
R9 and R10 are identical or different and independently from each other H or methyl, and
when R34 is SO₂ then R40 is CH₂;

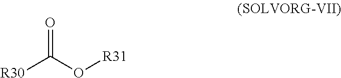

R30 is H or C₁₋₄ alkyl, and
R31 is C₁₋₄ alkyl;
   preferably,
R30 is H or C₁₋₂ alkyl, and
R31 is C₁₋₄ alkyl;

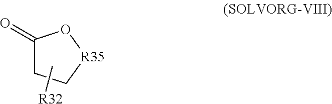

R32 is H or C₁₋₂ alkyl, and
R35 is CH₂, CH₂CH₂ or N—C₁₋₄ alkyl;
   preferably,
R32 is H, and
R35 is CH₂, CH₂CH₂ or N—CH₃;

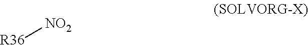

R36 is C₁₋₂ alkyl or phenyl;
   preferably,
R36 is methyl or phenyl;

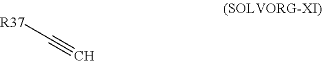

R37 is C₂₋₇ alkyl or phenyl;
   preferably,
R37 is ethyl, n-propyl, iso-propyl, butyl, pentyl, heptyl or phenyl;

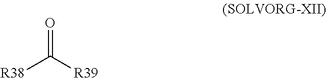

R38 and R39 are identical or different and independently from each other C₁₋₄ alkyl;
preferably, R38 and R39 have together 4 or 7 C atoms;
more preferably, R38 and R39 have together 4 or 6 C atoms;
even more preferably, R38 and R39 have together 5 or 6 C atoms.

Preferably, the carbonate-based solvent is for example ethylene carbonate, propylene carbonate, butylene carbonate, dimethyl carbonate, ethyl methyl carbonate and diethyl carbonate.

Preferably, the aliphatic ether-based solvent is for example dimethoxymethane, 1,2-dimethoxyethane, tetrahydrofuran, 2-methyltetrahydrofuran, 1,3-dioxane, 4-methyl-1,3-dioxolan, cyclopentyl methyl ether, dipropylether, diethylether, methyl-t-butyl ether, tert-amyl methyl ether, diethylene glycol dimethyl ether, diethylene glycol diethyl ether and triethylene glycol dimethyl ether.

Preferably, the ester-based solvent is for example methyl formate, methyl acetate, ethyl acetate, n-propyl acetate, isopropyl acetate, butyl acetate, methyl propionate, gamma-butyrolactone and gamma-valerolactone.

Preferably, the amide-based solvent is for example N-methyl oxazolidinone.

Preferably, the nitro-based solvent is for example nitromethane and nitrobenzene.

Preferably, the sulfur-based solvent is for example sulfolane and 3-methylsulfolane.

Preferably, the nitrile-based solvent is for example propionitrile, isobutyronitrile, butyronitrile, valeronitrile, capronitrile, caprylnitrile and benzonitrile.

Preferably, keton-based solvent is for example 3,3-dimethyl-2-butanone and 2,4-dimethyl-3-pentanone.

More preferably, SOLVORG is selected from the group consisting of ethylene carbonate, propylene carbonate, butylene carbonate, dimethyl carbonate, ethyl methyl carbonate, diethyl carbonate,
dimethoxymethane, 1,2-dimethoxyethane, tetrahydrofuran, 2-methyltetrahydrofuran, 1,3-dioxane, 4-methyl-1,3-dioxolan, cyclopentyl methyl ether, diisopropylether, diethylether, methyl-t-butyl ether, tert-amyl methyl ether, diethylene glycol dimethyl ether, diethylene glycol diethyl ether, triethylene glycol dimethyl ether,
ethyl acetate, n-propyl acetate, isopropyl acetate, butyl acetate, methyl propionate, gamma-butyrolactone, gamma-valerolactone,
N-methyl oxazolidinone,
propionitrile, isobutyronitrile, butyronitrile, valeronitrile, capronitrile, caprylnitrile, benzonitrile, 3,3-dimethyl-2-butanone, 2,4-dimethyl-3-pentanone, and mixtures thereof.

Even more preferably, SOLVORG is selected from the group consisting of ethylene carbonate, propylene carbonate, butylene carbonate, dimethyl carbonate, ethyl methyl carbonate, diethyl carbonate,
dimethoxymethane, 1,2-dimethoxyethane, tetrahydrofuran, 2-methyltetrahydrofuran, 1,3-dioxane, cyclopentyl methyl ether, diisopropylether, diethylether, methyl-t-butyl ether, tert-amyl methyl ether,
ethyl acetate, n-propyl acetate, isopropyl acetate, butyl acetate, methyl propionate, propionitrile, isobutyronitrile, butyronitrile, valeronitrile, 3,3-dimethyl-2-butanone, 2,4-dimethyl-3-pentanone, and mixtures thereof.

Especially, SOLVORG is selected from the group consisting of tetrahydrofuran, 2-methyltetrahydrofuran, diisopropylether, diethylether, methyl-t-butyl ether, tert-amyl methyl ether,
ethyl acetate, isopropyl acetate, butyl acetate, methyl propionate,
propionitrile, isobutyronitrile, butyronitrile, valeronitrile,
   3,3-dimethyl-2-butanone, 2,4-dimethyl-3-pentanone, and mixtures thereof.

More especially, SOLVORG is selected from the group consisting of THF, MeTHF, diethylether, diisopropylether, methyl-t-butyl ether, ethyl acetate, butyl acetate, valeronitrile, 3,3-dimethyl-2-butanone, and 2,4-dimethyl-3-pentanone.

Even more especially, SOLVORG is selected from the group consisting of THF, MeTHF, diethylether, diisopropylether, methyl-t-butyl ether, ethyl acetate, tert-butyl acetate, valeronitrile, 3,3-dimethyl-2-butanone, and 2,4-dimethyl-3-pentanone.

In particular, SOLVORG is selected from the group consisting of THF, MeTHF, methyl-t-butyl ether, ethyl acetate, tert-butyl acetate, valeronitrile, 3,3-dimethyl-2-butanone, and 2,4-dimethyl-3-pentanone.

More in particular, SOLVORG is methyl-t-butyl ether or butyl acetate or valeronitrile.

Even more in particular, SOLVORG is methyl-t-butyl ether or tert-butyl acetate.

Preferably, SOLVORG has a low boiling point.

Preferably, the weight ratio of MIXWAT:SOLVORG is from 0.5:1 to 15:1, more preferably from 1:1 to 10:1, even more preferably from 3:1 to 8:1.

EXTR can be done more than once, preferably 1, 2, 3, 4 or 5 times, more preferably 1, 2 or 3 times, even more preferably 1 or 2 times.

EXTR can be done batch wise or in a continuous way, preferably EXTR is done continuously, that is with a continuous extraction method. Continuous extraction methods are well known to the skilled person, for example counter current process or cross-flow process.

If EXTR is done more than once then the organic phases obtained from each extraction are combined after EXTR.

Any solution of a substance, that occurs in the process, such as MIXWAT or SOLCOMP1, can be filtered to remove insoluble impurities. Such filtration is known to the skilled person, typical mesh sizes are from 0.1 micrometer to 10 micrometer.

Any organic solution of a substance, that occurs in the process, such as SOLCOMP1, can be purified by extraction with water, this can be done once or more than once. This can be done for removal of water soluble impurities.

Compound of formula (I) can be isolated and purified by methods well-known to those skilled in the art. These methods include extraction, distillation, evaporation, membrane assisted separation, such as membrane assisted pervaporation or membrane assisted filtration; preferably isolation and purification is done by using a film evaporator, wiped film evaporator, falling film evaporation, distillation, rectification, flash distillation or short path distillation; more preferably a wiped film evaporator.

Preferably, any water is evaporated or distilled off, water can also be separated by a membrane separation.

Preferably, any SOLVORG is removed or at least partially removed by evaporation or by distillation.

Another subject of the invention is a method for preparation of a compound of formula (V);

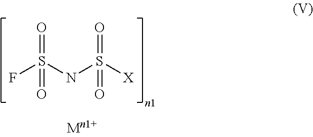

n1 is 1, 2 or 3;
M is selected from the group consisting of alkaline metal, alkaline earth metal and Al;
the method comprises STEP1 and a step STEP2;
with STEP1 as defined herein, also with all its embodiments;
in STEP2 the H of compound of formula (I) is exchanged against M.
STEP2 is done after STEP1.

The method does not necessarily comprise STEPMIX or STEPEXTR; in one embodiment, the method does not comprise STEPMIX; in another embodiment, the method does not comprise STEPEXTR; in yet another embodiment the method does not comprise STEPMIX and does not comprise STEPEXTR.

Preferably,

n1 is 1 in case that M is an alkaline metal;
n1 is 2 is case that M is an alkaline earth metal; and
n1 is 3 in case that M is Al.

Preferably, M is selected from the group consisting of Na, K, Li, Mg, and Al;

more preferably, M is selected from the group consisting of Na, K, and Li;
even more preferably, M is Na or Li;
especially, M is Li.

Specific embodiments of compound of formula (V) are compound of formula (5) and compound of formula (5-CF3).

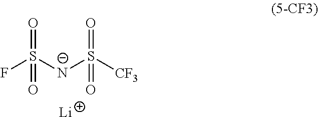 [SMILES: O=S(=O)(F)[N-]S(=O)(=O)C(F)(F)F ; [Li+]]

Another subject of the invention is a method for preparation of a compound of formula (V); the method comprises STEP1, STEPMIX, STEPEXTR and STEP2;
wherein STEP1, STEPMIX, STEPEXTR and STEP2 are as defined herein, also with all their embodiments.

Another subject of the invention is a method for preparation of a compound of formula (I-AMI);

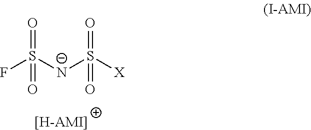

compound AMI is selected from the group consisting of N(R100)(R200)R300 and N(R400)R500;
R100, R200, R300 are identical or different and are selected from the group consisting of H, C₁₋₆ alkyl and halogenated C₁₋₆ alkyl; or
R100 and R200 form together with the N a saturated 5, 6, 7 or 8 membered heterocyclic ring RINGA;
R400 and R500 form together with the N an unsaturated 5, 6, 7 or 8 membered heterocyclic ring RINGB;
RINGA and RINGB can have 1 or 2 additional endocyclic heteroatoms selected from the group consisting of N, O and S;
RINGA and RINGB are unsubstituted or substituted by 1, 2 or 3 identical or different substituents selected from the group consisting of C₁₋₆ alkyl, halogenated C₁₋₆ alkyl, C₁₋₆ alkoxy and halogen;
   the method comprises STEP1 and a step STEP2-1;
   in STEP2-1 the H of compound of formula (I) is exchanged against H-AMI;
   with STEP1 as defined herein, also with all its embodiments.

STEP2-1 is done after STEP1.

The exchange of the H of compound of formula (I) against H-AMI in STEP2-1 provides compound of formula (I-AMI).

H-AMI is the protonated form of AMI.

Preferably,
R100, R200, R300 are identical or different and are selected from the group consisting of H, C₁₋₄ alkyl and halogenated C₁₋₄ alkyl; or
R100 and R200 form together with the N a saturated 5 or 6 membered heterocyclic ring RINGA;
R400 and R500 form together with the N an unsaturated 5 or 6 membered heterocyclic ring RINGB;
RINGA and RINGB can have 1 additional endocyclic heteroatom selected from the group consisting of N, O and S;
RINGA and RINGB are unsubstituted or substituted by 1, 2 or 3 identical or different substituents selected from the group consisting of C₁₋₄ alkyl, halogenated C₁₋₄ alkyl, C₁₋₄ alkoxy, F, Cl and Br;
more preferably,
R100, R200, R300 are identical or different and are selected from the group consisting of H, C₁₋₄ alkyl and halogenated C₁₋₄ alkyl; or
R100 and R200 form together with the N a saturated 5 or 6 membered heterocyclic ring RINGA;
R400 and R500 form together with the N an unsaturated 5 or 6 membered heterocyclic ring RINGB;
RINGA and RINGB can have 1 additional endocyclic N atom;
RINGA and RINGB are unsubstituted or substituted by 1, 2 or 3 identical or different substituents selected from the group consisting of C₁₋₂ alkyl, halogenated C₁₋₂ alkyl, C₁₋₂ alkoxy, F and Cl;
   even more preferably,
R100, R200, R300 are identical or different and are selected from the group consisting of H, C₁₋₄ alkyl and halogenated C₁₋₄ alkyl;
especially,
R100, R200, R300 are identical or different and are selected from the group consisting of H, C₁₋₄ alkyl and halogenated C₁₋₄ alkyl;
with the proviso that only one of the substituents can be H.

Particular embodiments of AMI are selected from the group consisting of NH₃, NH₂CH₃, NH(CH₃)₂, N(CH₃)₃, N(H₂)CH₂CH₃, NH(CH₂CH₃)₂, TEA, pyrrolidine, piperidine, pyrrol, pyrazol, imidazol, and pyridine;
the pyrrolidine, piperidine, pyrrol, pyrazol, imidazol, and pyridine are unsubstituted or substituted by 1 or 2 substituents selected from the group consisting of C₁₋₂ alkyl, F and Cl;
more particular embodiments of AMI are selected from the group consisting of NH₃, NH₂CH₃, NH(CH₃)₂, N(CH₃)₃, N(H₂)CH₂CH₃, NH(CH₂CH₃)₂, TEA, pyrrolidine, piperidine, pyrrol, pyrazol, imidazol, and pyridine;
the pyrrolidine, piperidine, pyrrol, pyrazol, imidazol, and pyridine are unsubstituted or substituted by 1 or 2 C₁₋₂ alkyl substituents;
even more particular embodiments of AMI are selected from the group consisting of NH₃, NH₂CH₃, NH(CH₃)₂, N(CH₃)₃, N(H₂)CH₂CH₃, NH(CH₂CH₃)₂, TEA, pyrrolidine, and piperidine;
the pyrrolidine and the piperidine are unsubstituted or substituted by 1 or 2 C₁₋₂ alkyl substituents;
very even more particular embodiments of AMI are selected from the group consisting of NH₃, NH₂CH₃, NH(CH₃)₂, N(CH₃)₃, N(H₂)CH₂CH₃, NH(CH₂CH₃)₂, and TEA.

Examples for substituted pyridine are 2-methyl-5-ethyl-pyridine, 2-picoline, 3-picoline and 4-picoline.

A specific embodiment of AMI is TEA.

Specific embodiments of compound of formula (I-AMI) are compound of formula (I-TEA), compound of formula (1-AMI), compound of formula (1-TEA), compound of formula (1-AMI-CF3) and compound of formula (1-TEA-CF3).

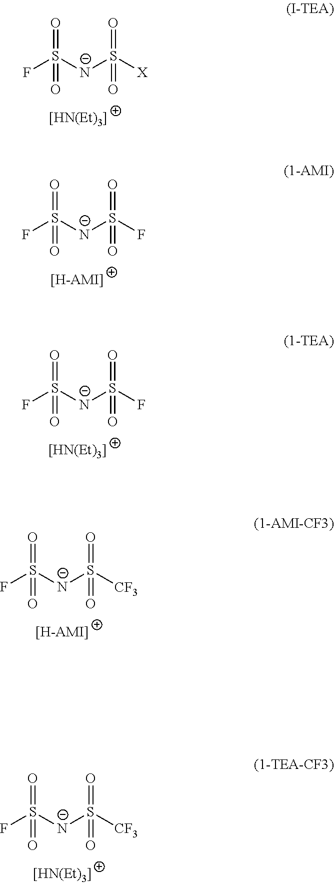

Another subject of the invention is a method for preparation of a compound of formula (I-AMI); the method comprises STEP1, STEPMIX, STEPEXTR and STEP2-1;
wherein STEP1, STEPMIX, STEPEXTR and STEP2-1 are as defined herein, also with all their embodiments.

Preferably, STEP2-1 comprises a reaction REAC2-1;

REAC2-1 is a reaction of compound of formula (I) with AMI.

The reaction product of REAC2-1 is compound of formula (I-AMI).

REAC2-1 can be done batch wise or in a continuous way, preferably REAC2-1 is done continuously.

Preferably, the molar amount of AMI is from 0.5 to 20 times, more preferably from 0.8 to 10 times, even more preferably from 0.9 to 5 times, even more preferably from 0.9 to 3 times, of the molar amount of compound of formula (I).

Om another preferred embodiment, the molar amount of AMI is from 1 to 10 times of the molar amount of compound of formula (I).

Preferably, REAC2-1 is done in aqueous medium, more preferably in water.

Preferably, the weight of water is from 0.5 to 50 times, more preferably from 1 to 25 times, even more preferably from 1 to 10 times, especially from 1 to 5 times, of the weight of compound of formula (I).

Preferably, REAC2-1 is done in water and at a pH of 1 to 12, more preferably of 2 to 12, even more preferably of 4 to 12, especially of 5 to 11, more especially of 6 to 11, even more especially of 6 to 10.

In another preferred embodiment, REAC2-1 is done in water and at a pH of 2 to 11, even more preferably of 3 to 11, especially of 3 to 10, more especially of 4 to 10.

Preferably, the pH is adjusted by the amount of AMI, The pH can further be adjusted by an addition of a base such a further AMI or alkaline metal hydroxide or alkaline earth metal hydroxide.

Preferably, REAC2-1 is done at a temperature TEMP2-1, TEMP2-1 is of from 0 to 100° C., more preferably from 5 to 80° C., even more preferably from 10 to 60° C., especially from 10 to 50° C., more especially from 20 to 50° C., in another more especial embodiment from 10 to 40° C.

Preferably, REAC2-1 is done at ambient pressure, it can also be done at a pressure of from ambient pressure to 10 bar, more preferably of from ambient pressure to 5 bar, even more preferably of from ambient pressure to 2.5 bar.

Preferably, the reaction time TIME2-1 of REAC2-1 is from 1 min to 2 h, more preferably from 2 min to 1.5 h, even more preferably 5 min to 1 h, especially from 5 min to 30 min.

In another preferred embodiment, TIME2-1 is from 5 min to 10 h.

Preferably, compound of formula (I) is charged to AMI.

Preferably, REAC2-1 is done in aqueous medium and compound of formula (I-AMI) is extracted after REAC2-1 from the aqueous medium by an extraction EXTR2-1 with a solvent EXTRSOLV2-1 or with SOLVORG, EXTR2-1 provides compound of formula (I-AMI) in form of a solution SOL-I-AMI, SOL-I-AMI is a solution of compound of formula (I-AMI) in EXTRSOLV2-1 or in SOLVORG;
EXTRSOLV2-1 forms a biphasic system with water;
with SOLVORG as defined herein, also with all its embodiments.

Therefore STEP2-1 comprises preferably EXTR2-1.

Preferably, EXTRSOLV2-1 is an organic solvent.

Preferably, EXTRSOLV2-1 has a low boiling point.

Preferably, EXTRSOLV2-1 is selected from the group consisting of carbonate-based solvent, aliphatic ether-based solvent, ester-based solvent, amide-based solvent, nitro-based solvent, sulfur-based solvent, nitrile-based solvent, and mixtures thereof.

Preferably, the carbonate-based solvent is for example ethylene carbonate, propylene carbonate, butylene carbonate, dimethyl carbonate, ethyl methyl carbonate and diethyl carbonate.

Preferably, the aliphatic ether-based solvent is for example dimethoxymethane, 1,2-dimethoxyethane, tetrahydrofuran, 2-methyltetrahydrofuran, 1,3-dioxane, 4-methyl-1,3-dioxolan, cyclopentyl methyl ether, diisopropylether, diethylether, methyl-t-butyl ether, diethylene glycol dimethyl ether, diethylene glycol diethyl ether and triethylene glycol dimethyl ether.

Preferably, the ester-based solvent is for example methyl formate, methyl acetate, ethyl acetate, n-propyl acetate, isopropyl acetate, butyl acetate, methyl propionate, gamma-butyrolactone and gamma-valerolactone.

Preferably, the amide-based solvent is for example N-methyl oxazolidinone.

Preferably, the nitro-based solvent is for example nitromethane and nitrobenzene.

Preferably, the sulfur-based solvent is for example sulfolane and 3-methylsulfolane.

Preferably, the nitrile-based solvent is for example propionitrile, isobutyronitrile, butyronitrile, valeronitrile, capronitrile, caprylnitrile and benzonitrile.

More preferably, EXTRSOLV2-1 is selected from the group consisting of ethylene carbonate, propylene carbonate, butylene carbonate, dimethyl carbonate, ethyl methyl carbonate, diethyl carbonate,
dimethoxymethane, 1,2-dimethoxyethane, tetrahydrofuran, 2-methyltetrahydrofuran, 1,3-dioxane, 4-methyl-1,3-dioxolan, cyclopentyl methyl ether, diisopropylether, diethylether, methyl-t-butyl ether, diethylene glycol dimethyl ether, diethylene glycol diethyl ether, triethylene glycol dimethyl ether,
methyl formate, methyl acetate, ethyl acetate, n-propyl acetate, isopropyl acetate, butyl acetate, methyl propionate, gamma-butyrolactone, gamma-valerolactone,
N-methyl oxazolidinone,
propionitrile, isobutyronitrile, butyronitrile, valeronitrile, capronitrile, caprylnitrile, benzonitrile, and mixtures thereof.

Even more preferably, EXTRSOLV2-1 is selected from the group consisting of ethylene carbonate, propylene carbonate, butylene carbonate, dimethyl carbonate, ethyl methyl carbonate, diethyl carbonate,
dimethoxymethane, 1,2-dimethoxyethane, tetrahydrofuran, 2-methyltetrahydrofuran, 1,3-dioxane, cyclopentyl methyl ether, diisopropylether, diethylether, methyl-t-butyl ether,
methyl acetate, ethyl acetate, n-propyl acetate, isopropyl acetate, butyl acetate, methyl propionate, propionitrile, isobutyronitrile, butyronitrile, valeronitrile, and mixtures thereof.

Especially, EXTRSOLV2-1 is selected from the group consisting of tetrahydrofuran, 2-methyltetrahydrofuran, diisopropylether, diethylether, methyl-t-butyl ether,
methyl acetate, ethyl acetate, isopropyl acetate, butyl acetate, methyl propionate,
propionitrile, isobutyronitrile, butyronitrile, valeronitrile, and mixtures thereof.

More especially, EXTRSOLV2-1 is selected from the group consisting of methyl acetate, ethyl acetate, isopropyl acetate, butyl acetate, methyl propionate,
propionitrile, isobutyronitrile, butyronitrile, valeronitrile, and mixtures thereof.

Even more especially, EXTRSOLV2 is valeronitrile.

In one preferred embodiment, the weight of EXTRSOLV2-1 or of SOLVORG is from 0.5 to 50 times, more preferably from 1 to 25 times, even more preferably from 1 to 10 times, of the weight of compound of formula (I).

In another preferred embodiment,
the weight ratio of [the aqueous medium from which compound of formula (I-AMI) is extracted after REAC2-1]:EXTRSOLV2-1, or
the weight ratio of [the aqueous medium from which compound of formula (I-AMI) is extracted after REAC2-1]:SOLVORG respectively,
is from 0.5:1 to 15:1, more preferably from 1:1 to 10:1, even more preferably from 3:1 to 8:1.

In one embodiment, SOL-I-AMI can be purified by extraction with water to remove water soluble impurities.

EXTR2-1 can be done more than once, preferably 1, 2, 3, 4 or 5 times, more preferably 1, 2 or 3 times.

EXTR2-1 can be done batchwise or in a continuous way, preferably EXTR is done continuously, that is with a continuous extraction method. Continuous extraction methods are well known to the skilled person, for example counter current process or cross-flow process.

If EXTR2-1 is done more than once then the organic phases are combined after EXTR2-1.

Compound of formula (I-AMI) can be isolated and purified by methods well-known to those skilled in the art. These methods include extraction, distillation, evaporation, and membrane assisted separation, such as membrane assisted pervaporation or membrane assisted filtration.

Preferably, STEP2 comprises a reaction REAC2;
REAC2 is a reaction of compound of formula (I) with a compound MET2;
MET2 is selected from the group consisting of M[OH]ₙ₁, alkaline metal carbonate, hydrogen carbonate and halide, and alkaline earth metal carbonate, hydrogen carbonate and halide;
the reaction product of REAC2 is compound of formula (V).

Preferably, MET2 is selected from the group consisting of hydroxide of Na, K, Li, Mg or Al, carbonate of Na, K, Li or Mg, hydrogen carbonate of Na, K, Li or Mg, and halide of Na, K, Li or Mg;
more preferably, MET2 is selected from the group consisting of hydroxide, carbonate, hydrogen carbonate and halide of Na, K or Li;
even more preferably, MET2 is selected from the group consisting of hydroxide, carbonate, hydrogencarbonate and halide of Li.

Hydroxide and halide are preferred embodiments of MET2, more preferably hydroxide.

MET2 can contain crystallization water.

MET2 can be used as such or as a mixture MIX-M, MIX-M is a mixture of MET2 with water, with SOLVORGANT, with EXTRSOLV2-1 or in a combination thereof;
preferably, MET2 can be used as a mixture MIX-M, MIX-M is a mixture of MET2 with water, with SOLVORGANT, with EXTRSOLV2-1 or in a combination thereof.

SOLVORGANT is selected from the group consisting of SOLVORG, ANTSOLV2 or a mixture thereof;
preferably, SOLVORGANT is SOLVORG;
with SOLVORG, EXTRSOLV2-1 and ANTSOLV2 as defined herein, also with all their embodiments.

ANTSOLV2 is a solvent with poor solubility of compound of formula (V).

ANTSOLV2 can be any solvent with poor solubility of compound of formula (V).

Preferably, ANTSOLV2 is an organic solvent.

Preferably, ANTSOLV2 is selected from the group consisting of aromatic hydrocarbon-based solvent, aliphatic hydrocarbon-based solvent, and mixtures thereof.

Preferably, the aromatic hydrocarbon-based solvent is unsubstituted or substituted by one or more of identical or different substituents selected from the group consisting of alkyl, alkoxy and halogen;
more preferably the aromatic hydrocarbon-based solvent is unsubstituted or substituted by 1, 2, 3 or 4 identical or different substituents selected from the group consisting of substituents C₁₋₄ alkyl, C₁₋₄ alkoxy, F, Cl, and Br.

Typical aromatic hydrocarbon-based solvent is unsubstituted or substituted benzene or naphthalene, preferably unsubstituted or substituted benzene, such as anisol, toluene, xylene, chlorobenzene or 1,2-dichlorobenzene.

Preferably, the aliphatic hydrocarbon-based solvent is unsubstituted, perhalogenated or substituted by one or more of identical or different substituents selected from the group consisting of alkyl, alkoxy and halogen;
more preferably the aliphatic hydrocarbon-based solvent is unsubstituted, perhalogenated or substituted by one or more of identical or different substituents selected from the group consisting of substituents C₁₋₄ alkyl, C₁₋₄ alkoxy, F, Cl, and Br;

Perhalogenated aliphatic hydrocarbon-based solvent is preferably a perfluorated or perchlorated or perchlorofluoro aliphatic hydrocarbon-based solvent.

Examples for aliphatic hydrocarbon-based solvent include paraffin, isoparaffin, alkylcyclohexane and cycloparaffin.

Example for paraffin include hexane, heptane, octane, decane, dodecane, undecane, tridecane and paraffin-containing mixed solvent.

Paraffin-containing mixed solvent is for example No. 0 SOLVENT L made by Nippon Oil Corporation.

Examples for isoparaffin include isohexane, isooctane, isododecane, isohexadecane, low molecular weight polybutene LV-7 (tetrahexamers: number-average molecular weight about 300), LV-50 (hexa-enneamers: number-average molecular weight about 450), LV-100 (octa-dodecamers: number-average molecular weight, about 500) [all made for example by Nippon Oil Corporation]; hydrogenated type polybutene OH (octa-heptamers: number-average molecular weight about 350), 5H (hexa-octamers: number-average molecular weight about 400), 10H-T (hepta-decamers: number-average molecular weight about 470) [all made for example by Idemitsu Kosan Co., Ltd.]; and commonly marketed isoparaffin-containing mixed solvents (e.g., IP SOLVENT of Idemitsu Kosan Co., Ltd., SHELLSOL T Series of Shell Chemicals Co., Ltd., ISOPER Series of Exxon Chemicals K.K.) and the like.

Isoparaffin is for example “Isopar™E” or “Isopar™G” manufactured by Exxon Mobil Corporation or “Marukasol R” manufactured by Maruzen Petrochemical Co., Ltd.

Examples of the cycloparaffin include alkylcyclohexane, commercially available naphthenic solvents, such as methyl cyclohexane, ethyl cyclohexane, SWACLEAN 150 (another name: a mixture of C₉ and C₁₀ alkyl cyclohexanes) [all made for example by Maruzen Petrochemical Co., Ltd.], Naphtesol Series and Cactus Solvent Series [all made for example by Nippon Oil Corporation] and the like.

Alkylcyclohexane is for example methyl cyclohexane, ethyl cyclohexane, C₉ cyclohexane, C₁₀ cyclohexane, C₁₁ cyclohexane.

It is also possible to use mixed solvents on the market which contain normal paraffin, isoparaffin and cycloparaffin.

Preferably, the aliphatic hydrocarbon-based solvent, which is perhalogenated or substituted by one or more of identical or different halogen atoms, and the aromatic hydrocarbon-based solvent, which is substituted by one or more of identical or different halogen atoms, include chlorobenzene, dichlorobenzene, trichlorobenzene, dichloromethane, and dichloroethane; more preferably chlorobenzene, 1,2-dichlorobenzene, dichloromethane and dichloroethane.

Especially, ANTSOLV2 is selected from the group consisting of 1,2,4-trimethylbenzene, tetralin, decaline, paraffin, isoparaffin, cycloparaffin, alkylcyclohexane, anisole, chlorobenzene, dichlorobenzene, trichlorobenzene, toluene, xylene, dichloromethane, dichloroethane, and mixtures thereof;
more especially, ANTSOLV2 is selected from the group consisting of 1,2,4-trimethylbenzene, alkylcyclohexane, anisole, chlorobenzene, dichlorobenzene, toluene, xylene, dichloromethane, dichloroethane, and mixtures thereof;
even more especially, ANTSOLV2 is selected from the group consisting of 1,2,4-trimethylbenzene, methyl cyclohexane, ethyl cyclohexane, C₉ cyclohexane, Cm cyclohexane, anisole, chlorobenzene, dichlorobenzene, toluene, xylene, dichloromethane, dichloroethane, and mixtures thereof;
in particular, ANTSOLV2 is selected from the group consisting of anisole, dichlorobenzene, toluene, xylene, dichloromethane, dichloroethane, and mixtures thereof;
more in particular, ANTSOLV2 is selected from the group consisting of 1,2-dichlorobenzene, dichloromethane, dichloroethane, and mixtures thereof.

Typical solvents used for washing are dichloromethane, pentane or hexane, preferably dichloromethane.

In one preferred embodiment, MIX-M is an aqueous solution, that is MET2 is used as an aqueous solution.

Preferably, MET2 is used as an aqueous solution of from 1 wt % to a saturated solution, even more preferably of from 3 wt % to a saturated solution, the wt % being based on the weight of the aqueous solution.

Preferably, in case that MET2 is LiOH, MET2 is used as an aqueous solution of from 1 to 13 wt %.

If MIX-M is a mixture of MET2 with water, then the weight of water, that is used to prepare MIX-M, is preferably from 0.5 to 50 times, more preferably from 1 to 25 times, even more preferably from 1 to 12.5 times, of the weight of MET2.

In another preferred embodiment, MIX-M is a mixture of MET2 with SOLVORGANT.

Preferably, the weight of SOLVORGANT, that is used to prepare MIX-M, is from 0.5 to 50 times, more preferably from 1 to 25 times, even more preferably from 1 to 12.5 times, of the weight of MET2.

Preferably, REAC2 is done at a temperature TEMP2, TEMP2 is of from −20 to 50° C., more preferably from −10 to 40° C., even more preferably from −5 to 40° C., especially from −4 to 30° C., more especially from −3 to 25.

Preferably, REAC2 is done at ambient pressure, it can also be done at a pressure of from ambient pressure to 10 bar, more preferably of from ambient pressure to 5 bar, even more preferably of from ambient pressure to 2.5 bar.

Preferably, the reaction time TIME2 of REAC2 is from 0.01 sec to 5 h, more preferably from 0.1 sec to 4 h, even more preferably 0.1 sec to 3 h.

In another preferred embodiment, TIME2 is from 5 min to 10 h.

REAC2 can be done batch wise or in a continuous way. Preferably, REAC2 is done continuously. For example a continuous device for REAC2 is an extraction centrifuge or an extraction column.

In one embodiment, compound of formula (I) is used in REAC2 in form of SOLCOMP1.

Therefore another subject of the invention is a method for preparation of compound of formula (V);
the method comprises STEP1 and STEP2;
STEP1 and STEP2 are as defined herein, also with all its embodiments;
wherein in REAC2 the MET2 is used as such or as a mixture MIX-M,
MIX-M is a mixture of MET2 with SOLVORGANT, with EXTRSOLV2-1 or with a combination of SOLVORGANT and EXTRSOLV2-1, or
MIX-M is a mixture of MET2 with SOLVORGANT, with EXTRSOLV2-1 or with a combination of SOLVORGANT and EXTRSOLV2-1, and with water, or
MIX-M is a mixture of MET2 with water;
in one preferred embodiment, MIX-M is a mixture of MET2 with SOLVORGANT, with EXTRSOLV2-1 or with a combination of SOLVORGANT and EXTRSOLV2-1; more preferably, MIX-M is a mixture of MET2 with SOLVORGANT, even more preferably, MIX-M is a mixture of MET2 with SOLVORG;
in another preferred embodiment, MIX-M is a mixture of MET2 with water;
in another preferred embodiment, MET2 is used as such.

Another subject of the invention is a method for preparation of a compound of formula (V); the method comprises STEP1, STEP2-1 and a step STEP2-2;
in STEP2-2 the H-AMI of compound of formula (I-AMI) is exchanged against M;
with STEP1, STEP2-1, AMI, H-AMI, compound of formula (I-AMI) and M as defined herein, also with all their embodiments.

Another subject of the invention is a method for preparation of a compound of formula (V); the method comprises STEP1, STEPMIX, STEPEXTR, STEP2-1 and STEP2-2;
wherein STEP1, STEPMIX, STEPEXTR, STEP2-1 and STEP2-2 are as defined herein, also with all their embodiments.

Preferably, compound of formula (I) is used in REAC2 or in REAC2-1 as obtained from STEP1 or from STEPEXTR, wherein STEP1 can include PUR1.

Preferably, when MET2 is used as such in REAC2, than a solvent such as SOLVORGANT or EXTRSOLV2-1 is added during or after REAC2.

Preferably, STEP2-2 comprises a reaction REAC2-2;
REAC2-2 is a reaction of compound of formula (I-AMI) with MET2;
with MET2 as defined herein, also with all its embodiments, also with all its forms of use.

The reaction product of REAC2-2 is compound of formula (V).

When n1 is 1, then preferably the molar amount of MET2 is from 1 to 100 times, more preferably from 1 to 50 times, even more preferably from 1 to 10 times, especially from 1 to 5 times, more especially from 1 to 3 times, even more especially from 1 to 2 times, in particular from 1 to 1.5, more in particular from 1 to 1.2, of the molar amount of compound of formula (I) in case of REAC2 or of the molar amount of compound of formula (I-AMI) in case of REAC2-2. The lower limit for the molar amount of MET2, in case that n1 is 1, can also be below 1 time of the molar amount of compound of formula (I) or of compound of formula (I-AMI) respectively, since in the end it is primarily a question of yield and costs, whether MET2 is used in excess or whether compound of formula (I) or compound of formula (I-AMI) respectively is used in excess. For example the lower limit for the molar amount of MET2, in case that n1 is 1, can also be for example 0.85 or 0.9 or 0.95 times of the molar amount of compound of formula (I) or of compound of formula (I-AMI) respectively, these lower limits can be combined with any of the mentioned upper limits.

When n1 is 2, then preferably, the molar amount of MET2 is from 0.5 to 50 times, more preferably from 0.5 to 25 times, even more preferably from 0.5 to 5 times, especially from 0.5 to 2.5 times, more especially from 0.5 to 1.5 times, even more especially from 0.5 to 1 times, of the molar amount of compound of formula (I) in case of REAC2 or of the molar amount of compound of formula (I-AMI) in case of REAC2-2. The lower limit for the molar amount of MET2, in case that n1 is 2, can also be below 0.5 time of the molar amount of compound of formula (I) or of compound of formula (I-AMI) respectively, since in the end it is primarily a question of yield and costs, whether MET2 is used in excess or whether compound of formula (I) or compound of formula (I-AMI) respectively is used in excess. For example the lower limit for the molar amount of MET2, in case that n1 is 2, can also be for example 0.35 or 0.4 or 0.45 times of the molar amount of compound of formula (I) or of compound of formula (I-AMI) respectively, these lower limits can be combined with any of the mentioned upper limits.

When n1 is 3, then preferably, the molar amount of MET2 is from 0.33 to 33 times, more preferably from 0.33 to 17.33 times, even more preferably from 0.33 to 3.33 times, especially from 0.33 to 1.733 times, more especially from 0.33 to 1 times, even more especially from 0.33 to 0.733 times, of the molar amount of compound of formula (I) in case of REAC2 or of the molar amount of compound of formula (I-AMI) in case of REAC2-2. The lower limit for the molar amount of MET2, in case that n1 is 3, can also be below 0.33 time of the molar amount of compound of formula (I) or of compound of formula (I-AMI) respectively, since in the end it is primarily a question of yield and costs, whether MET2 is used in excess or whether compound of formula (I) or compound of formula (I-AMI) respectively is used in excess. For example the lower limit for the molar amount of MET2, in case that n1 is 3, can also be for example 0.25 or 0.3 times of the molar amount of compound of formula (I) or of compound of formula (I-AMI) respectively, these lower limits can be combined with any of the mentioned upper limits.

Preferably, REAC2-2 is done at a temperature TEMP2-2, TEMP2-2 is of from −20 to 100° C., more preferably from −10 to 80° C., even more preferably from 0 to 60° C., especially from 10 to 60° C., more especially from 20 to 50° C.

In another preferred embodiment, REAC2-2 is done at a temperature TEMP2-2, TEMP2-2 is of from −20 to 50° C., more preferably from −10 to 40° C., even more preferably from −5 to 40° C., especially from −4 to 30° C., more especially from −3 to 25.

Preferably, REAC2-2 is done at ambient pressure, it can also be done at a pressure of from ambient pressure to 10 bar, more preferably of from ambient pressure to 5 bar, even more preferably of from ambient pressure to 2.5 bar.

Preferably, the reaction time TIME2-2 of REAC2-2 is from 0.01 sec to 5 h, more preferably from 0.1 sec to 4 h, even more preferably 0.1 sec to 3 h.

In another preferred embodiment, TIME2-2 is from 5 min to 10 h.

REAC2-2 can be done batch wise or in a continuous way. Preferably, REAC2-2 is done continuously. For example a continuous device for REAC2-2 is an extraction centrifuge or an extraction column.

Preferably, compound of formula (I-AMI) is used in REAC2-2 as obtained from REAC2-1 or EXTR2-1, more preferably as obtained from EXTR2-1.

After EXTR2-1 and preferably before REAC2-2, the solution of compound of formula (I-AMI) in EXTRSOLV2-1 can be mixed with NH₃;
preferably, 0.0001 to 100 times, more preferably, 0.001 to 50 times, even more preferably 0.01 to 50 times, of the molar amount of compound of formula (I-AMI), of NH₃ is used;
in another preferred embodiment, NH₃ is used in such an amount that the pH after addition of NH₃ is 1 to 12, more preferably 2 to 12, even more preferably 4 to 12, especially of 5 to 11, more especially 6 to 11.

Compound of formula (V) can be isolated and purified by methods well-known to those skilled in the art. These methods include extraction, distillation, evaporation, membrane assisted separation, such as membrane assisted pervaporation or membrane assisted filtration, further crystallization or precipitation; any crystallization or precipitation can be done with the help of a solvent wherein compound of formula (V) is only poorly soluble.

Preferably, any water or any AMI can be removed or at least partially removed by evaporation or by distillation, water can also be removed and separated or at least partially removed and separated by membrane separation.

Preferably, any solvent such as SOLVORG, EXTRSOLV2-1 or any ANTSOLV2 can be removed or at least partially removed by evaporation or by distillation.

Preferably, compound of formula (I-AMI) is used in REAC2-2 in form of SOL-I-AMI.

Compound of formula (V) is preferably obtained from REAC2-2 in form of a mixture SOLCOMP5-2-2, SOLCOMP5-2-2 is a mixture of compound of formula (V) in EXTRSOLV2-1, in SOLVORGANT, or in a combination thereof.

Compound of formula (V) is preferably obtained from REAC2 in form of a solution or mixture SOLCOMP5, SOLCOMP5 is a solution or a mixture of compound of formula (V) in SOLVORGANT, that is in SOLVORG, in ANTSOLV2 or in a combination thereof, preferably SOLCOMP5 has the form of a solution.

When MET2 is used as an aqueous solution or in form of a mixture with water or in form of an hydroxide, or when MET2 contains crystallization water, then two layers can be formed in REAC2 or in REAC2-2, that means a biphasic system can be formed, an organic layer in form of SOLCOMP5 or of SOLCOMP5-2-2 and an aqueous layer.

Preferably, any aqueous layer is separated from said organic layer in form of SOLCOMP5 or of SOLCOMP5-2-2, before compound of formula (V) is isolated.

Also water can be present in SOLCOMP5 or in SOLCOMP5-2-2, for example depending in the solubility of water in SOLVORGANT or in EXTRSOLV2-1.

The nature of SOLVORG and the nature of ANTSOLV2, that is the nature of SOLVORGANT, and the nature of EXTRSOLV2-1, especially in view of their solubility with water, determines the amount of SOLVORG, of ANTSOLV2 and of EXTRSOLV2-1 in order to obtain preferably a biphasic system after REAC2 or after REAC2-2 respectively, for example in case that MET2 is used as an aqueous solution or as a mixture with water, preferably a biphasic system after REAC2 or after REAC2-2 is desired. Additional SOLVORG, ANTSOLV2 or EXTRSOLV2-1 or additional water can be added after REAC2 or after REAC2-2 respectively, in order to obtain such a biphasic system.

For example, the weight ratio [of an aqueous solution of MET2 or of a mixture of MET2 with water]: [SOLVORG or ANTSOLV2 or SOLVORGANT or EXTRSOLV2-1], is from 0.05:1 to 1:1, more preferably from 0.1:1 to 0.8:1, even more preferably from 0.1:1 to 0.6:1.

For example, the weight ratio water:[SOLVORG or ANTSOLV2 or SOLVORGANT or EXTRSOLV2-1 or a combination thereof], is from 0.05:1 to 1:1, more preferably from 0.1:1 to 0.8:1, even more preferably from 0.1:1 to 0.6:1.

After REAC2 and REAC2-2 respectively, SOLCOMP5 and SOLCOMP5-2-2 respectively can be purified by extraction with water, this can be done once or more than once. This can be done for removal of water soluble impurities.

In principle, any solution of a substance that occurs in any of the methods, such as a solution of compound of formula (I), of compound of formula (I-AMI) or of compound of formula (V), in an organic solvent, such as in SOLVORG, in ANTSOLV2, in SOLVORGANT or in EXTRSOLV2-1,
can be purified by extraction with water, this can be done once or more than once or it can be done in a continuous way. This can for example be done for removal of water soluble impurities.

For isolation of compound of formula (V) from SOLCOMP5 or from SOLCOMP5-2-2, in the latter case for example for isolation of compound of formula (V) from its solution in EXTRSOLV2-1, preferably any water and any AMI are removed by evaporation, preferably under reduced pressure. The evaporation is preferably done in form of a distillation. Preferably, also any SOLVORG or any ANTSOLV2 or any EXTSOLV2-1 is removed at least partly by evaporation, preferably under reduced pressure. Preferably a SOLCOMP5 or a SOLCOMP5-2-2 in concentrated form is thereby obtained, in the latter case for example a concentrated solution of compound of formula (V) in EXTRSOLV2-1. Preferably, the concentration of compound of formula (V) in said concentrated SOLCOMP5 or in said concentrated SOLCOMP5-2-2, for example in said concentrated solution of compound of formula (V) in EXTRSOLV2-1, is from 30 to 55% by weight, more preferably from 35 to 50% by weight, of compound of formula (V), the % by weight being based on the total weight of said concentrated SOLCOMP5, of said concentrated SOLCOMP5-2-2 or of said concentrated solution of compound of formula (V) in EXTRSOLV2-1.

Any solution, such as a solution
of compound of formula (I) in SOLVORG, in ANTSOLV2, in SOLVORGANT, in EXTRSOLV2-1, in water, or in a combination thereof,
of compound of formula (I-AMI) in SOLVORG, in ANTSOLV2, in SOLVORGANT, IN EXTRSOLV2-1, in water, or in a combination thereof; or
of compound of formula (V) in SOLVORG, in ANTSOLV2, in SOLVORGANT, in EXTRSOLV2-1, in water, or in a combination thereof,
can be filtered to remove insoluble impurities. Such filtration is known to the skilled person, typical mesh sizes are from 0.1 micrometer to 10 micrometer.

Preferably, compound of formula (V) is precipitated
from SOLCOMP5, that is the solution or a mixture of compound of formula (V) in SOLVORG or in ANTSOLV2, that is in SOLVORGANT;
or from SOLCOMP5-2-2;
by a precipitation PRECIP2.

Preferably, before PRECIP2 or as part of PRECIP2, SOLCOMP5 or SOLCOMP5-2-2 is concentrated, preferably by removal of part of SOLVORG, of ANTSOLV2, that is of SOLVORGANT, or of EXTRSOLV2-1, by distillation.

Preferably PRECIP2 is done by a distillation DIST2 or by a crystallization CRYST2 or by a combination of both.

Preferably, in PRECIP2 ANTSOLV2 is added.

ANTSOLV2 can for example be added in PRECIP2 when SOLCOMP5 is a solution or a mixture of compound of formula (V) in SOLVORG; ANTSOLV2 can of course also be added in PRECIP2 when ANTSOLV2 is already present in SOLCOMP5, this can depend on the amount of ANTSOLV2, that may be present in SOLCOMP5 in order to facilitate crystallization.

Preferably, any ANTSOLV2 is added either before, during or after DIST2 or CRYST2, preferably before or during DIST2 or before or during CRYST2.

Cooling can be used in CRYST2.

Compound of formula (V), that was precipitated in PRECIP2, can be isolated by filtration, washing and drying.

Any distillation is preferably done at a temperature of 70° C. or below, more preferably of 60° C. or below.

Any crystallization is preferably enhanced by cooling, preferably by cooling to a temperature of 30° C. or below.

Any mother liquor obtained from the filtration of compound of formula (V) after PRECIP2 is preferably recycled into PRECIP2.

Any of the methods disclosed herein can also comprise a step STEPDISSOL-S1, in STEPDISSOL-S1 compound of formula (I), preferably as obtained from STEP1, is dissolved in SOLVORG to provide a solution SOLCOMP1-S1, SOLCOMP-S1 is a solution of compound of formula (I) in SOLVORG;
with STEP1 and SOLVORG as defined herein, also with all their embodiments.

Preferably, the weight of SOLVORG, that is used to dissolve in STEPDISSOL-S1 compound of formula (I), is from 0.5 to 50 times, more preferably from 1 to 25 times, even more preferably from 1 to 10 times, of the weight of compound of formula (I).

Another subject of the invention is a method for preparation of compound of formula (I); the method comprises STEP1 and STEPDISSOL-S1;
with STEP1 and STEPDISSOL-S1 as defined herein, also with all their embodiments.

STEPDISSOL-S1 is done after STEP1.

The method does not necessarily comprise STEPMIX or STEPEXTR; in one embodiment, the method does not comprise STEPMIX; in another embodiment, the method does not comprise STEPEXTR; in yet another embodiment the method does not comprise STEPMIX and does not comprise STEPEXTR.

Another subject of the invention is a method for preparation of compound of formula (V); the method comprises STEP1, STEPDISSOL-S1 and STEP2;
with STEP1, STEPDISSOL-S1 and STEP2 as defined herein, also with all their embodiments.

STEPDISSOL-S1 is done after STEP1.

STEP2 is done after STEPDISSOL-S1.

The method does not necessarily comprise STEPMIX or STEPEXTR; in one embodiment, the method does not comprise STEPMIX; in another embodiment, the method does not comprise STEPEXTR; in yet another embodiment the method does not comprise STEPMIX and does not comprise STEPEXTR.

STEP2 is done after STEP1, after STEPMIX, after STEPEXTR or after STEPDISSOL-S1.

SOLCOMP1-S1 can be treated in an analogous way as described herein for SOLCOMP1;

compound of formula (I) can be used for REAC2 in form of SOLCOMP1-S1 in an analogous way as describe herein, also with all embodiments, for the use of SOLCOMP1 for REAC2.

So therefore, anywhere, where SOLCOMP1 is used, also SOLCOMP1-S1 can be used instead of SOLCOMP1

A preferred embodiment of the method for preparation of compound of formula (V) comprises the following operations, preferably in the given sequence, preferably starting with compound of formula (I) as obtained from STEP1; optionally STEP1 comprises PUR1:
STEPMIX providing MIXWAT;
STEPEXTR providing SOLCOMP1;
optionally an extraction of SOLCOMP1 with water for removal of water soluble impurities;
STEP2 with REAC2 with SOLCOMP1 providing SOLCOMP5;
optionally a separation of water from SOLCOMP5 at any stage, preferably after REAC2, preferably by membrane separation for reducing the amount of water;
optionally concentration of SOLCOMP5 by removal of part of SOLVORG and of at least part of any water that may be present, by distillation or evaporation, preferably with a thin film evaporator;
optionally a filtration of SOLCOMP5 at any stage, preferably of the concentrated SOLCOMP5, for removal of insoluble impurities;
optionally crystallization by removal of SOLVORG by distillation or evaporation and preferably by cooling, optionally with addition of ANTSOLV2, the addition of ANTSOLV2 is preferably done either during the distillation or after the distillation;
optionally isolation by filtration.

Another preferred embodiment of the method for preparation of compound of formula (V) comprises the following operations, preferably in the given sequence, starting with compound of formula (I), preferably starting with compound of formula (I) as obtained in STEP1; optionally STEP1 comprises PUR1:
STEPDISSOL-S1 providing SOLCOMP1-S1;
optionally an extraction of SOLCOMP1-S1 with water for removal of water soluble impurities;
STEP2 with REAC2 with SOLCOMP1-S1 providing SOLCOMP5;
optionally a separation of water from SOLCOMP5 at any stage, preferably after REAC2, preferably by membrane separation for reducing the amount of water;
optionally concentration of SOLCOMP5 by removal of part of SOLVORG and of at least part of any water that may be present, by distillation or evaporation, preferably with a thin film evaporator;
optionally a filtration of SOLCOMP5 at any stage, preferably of the concentrated SOLCOMP5, for removal of insoluble impurities;
optionally crystallization by removal of SOLVORG by distillation or evaporation and preferably by cooling, optionally with addition of ANTSOLV2, the addition of ANTSOLV2 is preferably done either during the distillation or after the distillation;
optionally isolation by filtration.

Another preferred embodiment of the method for preparation of compound of formula (V) comprises the following operations, preferably in the given sequence, starting with compound of formula (I), preferably starting with compound of formula (I) as prepared in STEP1; optionally STEP1 comprises PUR1:
STEP2 providing SOLCOMP5, wherein MET2 is used in form of MIX-M or as such, MIX-M is a mixture of MET2 with SOLVORGANT; preferably, SOLVORGANT is SOLVORG; when MET2 is used as such in REAC2, than preferably a solvent such as SOLVORGANT is added during or after REAC2.
optionally an extraction of SOLCOMP5 with water for removal of water soluble impurities;
optionally a separation of water from SOLCOMP5, preferably by membrane separation, for reducing the amount of water;
optionally a concentration of SOLCOMP5, preferably by removal of at least part of SOLVORGANT and of at least part of any water that may be present, by distillation or evaporation, preferably with a thin film evaporator;
optionally a filtration of SOLCOMP5 at any stage, preferably of the concentrated SOLCOMP5, for removal of insoluble impurities;
optionally crystallization by removal of SOLVORGANT by distillation or evaporation and preferably by cooling, optionally with addition of ANTSOLV2, the addition of ANTSOLV2 is preferably done either during the distillation or evaporation or after the distillation or evaporation;
optionally isolation by filtration.

Preferably, REAC1-1 is done at a pressure PRESSURE1-1.

Preferably, PRESSURE1-1 is at least ambient pressure, more preferably at least 2 bar, even more preferably at least 5 bar, very even more preferably at least 10 bar, very, very even more preferably at least 20 bar, especially at least 30 bar, more especially at least 40 bar, even more especially at least 45 bar, very even more especially at least 50 bar, very, very even more especially at least 55 bar, in particular at least 60 bar, more in particular at least 65 bar, even more in particular at least 70 bar, very even more in particular at least 75 bar, very, very even more in particular at least 80 bar.

The upper limit of the pressure is mainly determined by the devices and their ability to provide and/or stand the pressure. Purely out of such considerations and without limiting the invention, PRESSURE1-1 is preferably up to 1000 bar, more preferably up to 750 bar, even more preferably up to 600 bar, especially up to 500 bar.

Any of the lower limits of PRESSURE1-1 can be combined with any of the upper limits of PRESSURE1-1;
preferably, PRESSURE1-1 is from ambient pressure to 1000 bar, more preferably from 2 to 1000 bar, even more preferably from 5 to 1000 bar, very even more preferably from 10 to 1000 bar, very, very even more preferably from 20 to 1000 bar, especially from 30 to 1000 bar, more especially from 40 to 1000 bar, even more especially from 45 to 1000 bar, very even more especially from 50 to 1000 bar, in particular from 55 to 1000 bar, more in particular from 60 to 1000 bar, even more in particular from 65 to 1000 bar, very even more in particular from 70 to 750 bar, very, very even more in particular from 75 to 600 bar, 3 times very even more in particular from 80 to 500 bar.

Preferably, REAC1-1 is done at a temperature TEMP1-1.

Preferably, TEMP1-1 is at least 80° C., more preferably at least 90° C., even more preferably at least 100° C., especially at least 110° C., more especially at least 120° C., even more especially at least 125° C., in particular at least 130° C., more in particular at least 135° C., even more in particular at least 140° C., very even more in particular at least 145° C., very, very even more in particular at least 150° C., very, very, very even more in particular at least 155° C., very, very, very, very even more in particular at least 160° C.

The upper limit of the temperature is mainly determined by the residence time of the components at elevated temperatures, the shorter the residence time the higher can be the temperature; and also be the resistance against corrosion of the chosen materials of the devices at elevated temperatures. Purely out of such considerations and without limiting the invention, TEMP1-1 can preferably be up to 300° C., more preferably up to 290° C., even more preferably up to 280° C., especially up to 270° C., more especially up to 260° C., even more especially up to 250° C., in particular up to 240° C., more in particular up to 230° C.

Preferably, TEMP1-1 is from 80 to 300° C., more preferably from 90 to 300° C., 100 to 300° C., even more preferably from 110 to 290° C., especially from 120 to 280° C., more especially from 130 to 280° C., even more especially from 130 to 280° C., in particular from 140 to 280° C., more in particular from 145 to 280° C., even more in particular from 150 to 250° C., very even more in particular from 150 to 230° C., very, very even more in particular from 155 to 230° C.

Any of the given minimum points, maximum points and/or ranges of TEMP1-1 can be combined with any of the given minimum points, maximum points and/or ranges of PRESSURE1-1.

Preferably, mixture of compound of formula (II) and HF is heated in a device DEVICE1-1 to TEMP1-1, REAC1-1 takes place in DEVICE1-1.

Preferably, TIME1-1 is the time, where the mixture is exposed to heating, preferably to TEMP1-1, preferably in DEVICE1-1. During TIME1-1 REAC1-1 takes place. TIME1-1 is therefore preferably a residence time and is preferably the residence time of the mixture in DEVICE1-1.

Preferably, TIME1-1 is from 1 min to 2 h, more preferably from 2 min to 1.5 h, even more preferably 5 min to 1 h, especially from 5 min to 30 min.

Preferably, HCl, that is produced in REAC1-1, is not removed selectively during REAC1-1 to produce compound of formula (I) in at least 80% yield;
more preferably, HCl, that is produced in REAC1-1, is not removed selectively during REAC1-1 to produce compound of formula (I) in higher yield;
even more preferably, HCl, that is produced in REAC1-1, is not removed selectively during REAC1-1.

In another preferred embodiment, HCl, that is produced in REAC1-1, is not removed selectively to produce compound of formula (I) in at least 80% yield;
more preferably, HCl, that is produced in REAC1-1, is not removed selectively to produce compound of formula (I) in higher yield;
even more preferably, HCl, that is produced in REAC1-1, is not removed selectively.

Preferably, the molar amount of HF is from 2 to 40 times, more preferably from 2 to 20 times, and even more preferably from 2 to 12.5 times, especially from 2 to 10 times, more especially from 2 to 5 times, even more especially from 2 to 4 times, in particular from 2 to 3 times, more in particular from 2 to 2.5 times, based on the molar amount of compound of formula (II).

In principle it is also possible to use the HF in substoichiometric amounts, that is below 2 equivalents, with respect to the molar amount of compound of formula (II). Naturally in such a case the yield will be lower with respect to compound of formula (II). But also this embodiment is comprised by the invention. Therefore also preferably, the molar amount of HF is from 0.1 to 40 times, more preferably from 0.2 to 40 times, and even more preferably from 0.5 to 40 times, especially 1 to 40 times, more especially 1.5 to 40 times, even more especially 1.75 to 40 times, based on the molar amount of compound of formula (II).

Preferably, at least one of the residues X1 and X2 is Cl or Br, more preferably Cl.

Preferably, the lower limit LOWLIMIT of the amount of HF is 1 equivalent based on the molar amount of compound of formula (II) in case that only one of the residues X1 and X2 is Cl, Br, or I;

LOWLIMIT is 2 equivalents in case that both residues X1 and X2 are identical or different and selected from the group consisting of Cl, Br, and I.

Preferably, the molar amount of HF is from LOWLIMIT to 40 times, more preferably from LOWLIMIT to 20 times, and even more preferably from LOWLIMIT to 12.5 times, especially from LOWLIMIT to 10 times, more especially from LOWLIMIT to 5 times, even more especially from LOWLIMIT to 4 times, in particular from LOWLIMIT to 3 times, more in particular from LOWLIMIT to 2.5 times, based on the molar amount of compound of formula (II).

In principle it is also possible to use the HF in substoichiometric amounts, that is below LOWLIMIT, with respect to the molar amount of compound of formula (II). Naturally in such a case the yield will be lower with respect to compound of formula (II). But also this embodiment is comprised by the invention. Therefore also preferably, the molar amount of HF is from 0.1 to 40 times, more preferably from 0.2 to 40 times, and even more preferably from 0.5 to 40 times, especially 1 to 40 times, more especially 1.5 to 40 times, even more especially 1.75 to 40 times, of LOWLIMIT, based on the molar amount of compound of formula (II).

Any of these lower ranges can be combined with any of the upper ranges given herein and vice versa.

In a preferred embodiment, STEP1 comprises two consecutive steps, a step STEP1-1 and a step STEP1-3;
in STEP1-1 a mixture MIXTURE1-1, MIXTURE1-1 is a mixture of compound of formula (II) and HF, is heated in a device DEVICE1-1 to TEMP1-1, REAC1-1 takes place in DEVICE1-1 resulting in a reaction mixture,
in STEP1-3 the reaction mixture from DEVICE1-1 passes through a device DEVICE1-3, DEVICE1-3 is a device for back pressure regulation.

Preferably, STEP1 comprises a third step, a step STEP1-2, which is done either before or after STEP1-3, preferably between STEP1-1 and STEP1-3, in STEP1-2 the reaction mixture from DEVICE1-1 passes through a device DEVICE1-2, DEVICE1-2 is a device for cooling the reaction mixture.

Preferably, the reaction mixture is cooled by the effects of DEVICE1-2 or of DEVICE1-3 or of a combination of DEVICE1-2 and DEVICE1-3 on the reaction mixture.

DEVICE1-1, DEVICE1-2 and DEVICE1-3 are continuously working devices.

Time TIME1-2 is a time, where the reaction mixture is cooled, preferably to a temperature TEMP1-2. Preferably, the cooling can be done by the action of DEVICE1-2, by the action of DEVICE1-3 or by the action of DEVICE1-2 and DEVICE1-3. TIME1-2 is therefore preferably a residence time and is preferably the residence time of the reaction mixture in DEVICE1-2 and/or in DEVICE1-3.

Preferably, TIME1-2 is from 0.1 sec to 2 h, more preferably from 0.5 sec to 1 h, even more preferably 1 sec to 30 min, especially from 10 sec to 30 min, more especially from 25 sec to 25 min, even more especially from 1 min to 25 min.

The cooling in STEP1-2 is preferably done to TEMP1-2, preferably, TEMP1-2 is from 0 to 150° C., more preferably from 10 to 100° C., even more preferably from 10 to 50° C., especially from 15 to 40° C., more especially from 15 to 30° C.

Preferably, the method comprises furthermore a step STEP1-4, STEP1-4 is done after STEP1-3, in STEP1-4 the reaction mixture from DEVICE1-3 passes through a device DEVICE1-4, DEVICE1-4 is a device for separating gaseous components from liquid components in the reaction mixture.

The byproduct of REAC1-1 is HCl.

Preferably, MIXTURE1-1 is fed into DEVICE0-1, during the passage through DEVICE1-1, the initially fed MIXTURE1-1 gradually is converted to the reaction mixture by REAC1-1.

Preferably, DEVICE1-1 is selected from the group consisting of tube, microreactor, shell and tube heat exchanger, plate heat exchanger and any common device which purpose is to exchange heat from a fluid;
more preferably it is a tube;
even more preferably it is a coiled tube.

Preferably, DEVICE1-2 is selected from the group consisting of tube, microreactor, shell and tube heat exchanger, plate heat exchanger and any common device which purpose is to exchange heat from a reaction mixture;
more preferably it is a tube;
even more preferably it is a coiled tube.

Especially, DEVICE1-1 and DEVICE1-2 are coiled tubes.

Preferably, DEVICE1-3 is a conventional back pressure regulating device.

Preferably, DEVICE1-4 a device capable of separating gaseous components from a liquid, any known device suitable for this purpose for can be used for this purpose, more preferably DEVICE1-4 is a vessel, a column or a cyclone.

The heating, preferably in DEVICE1-1, can be done be any known means, preferably it is done by electric heating or by heating with a fluid heat carrier.

Cooling, preferably in DEVICE1-2, can be done be any known means, preferably it is done by a fluid cooling medium.

Depending on the scale of the reaction and thereby on the scale of the apparatus, wherein the method is done, the cooling of the reaction mixture is done not only by the effect of DEVICE1-2 on the reaction mixture, i.e. it is not only during the passage of the reaction mixture through DEVICE1-2, but additionally the effects of DEVICE1-3 on the reaction mixture, i.e. the passage through DEVICE1-3 contributes to the cooling. This is especially the case when the scale of the reaction is rather small, e.g. when the method is done on lab scale, whereas in case where the method is done on a production scale the cooling will usually primarily be done during the passage through DEVICE1-2.
In another embodiment, especially on production scale, cooling can also be achieved by the expansion and pressure release affected by DEVICE1-3.
Also a combination of cooling during the passage through DEVICE1-2 with a cooling by expansion effected by DEVICE1-3 is possible.

Therefore when the description refers to a cooling in DEVICE1-2, this reference also comprises cooling in DEVICE1-3 and cooling in both devices DEVICE1-2 and DEVICE1-3.

Preferably, heating in DEVICE1-1 and cooling in DEVICE1-2 is realized in form of a tube-in-tube set up, in form of a tube-in-container set up, in form of a shell and tube heat exchanger, plate heat exchanger or any common device which purpose is to exchange heat from a mixture or a reaction mixture;
more preferably, heating in DEVICE1-1 and cooling in DEVICE1-2 is realized in form of a tube-in-tube set up or in form of a tube-in-container set up.

REAC1-1 is triggered, preferably in DEVICE1-1, by the heating of the mixture to TEMP1-1, preferably in the DEVICE1-1.

The PRESSURE1-1 in DEVICE1-1 and preferably in DEVICE1-2 is controlled and maintained by the DEVICE1-3.

HF and compound of formula (II) can be fed into the DEVICE1-1 as a premixed mixture or can be fed into the DEVICE1-1 separately and are mixed in DEVICE1-1.

For the purpose of mixing of HF and compound of formula (II) before or in DEVICE1-1 any suitable installation for mixing can be used, which are known in the state of the art, such as a common branch connection, e.g. a T or Y piece, or a static mixing device.

Preferably the heating to TEMP1-1 in DEVICE1-1 is done only when both HF and compound of formula (II) are present in DEVICE1-1.

The feeding of HF and compound of formula (II), either separately or in form of a mixture, is done by a device DEVICE1-0.

DEVICE1-0 is a pressuring device conventionally used to convey a fluid against pressure, such as a pump. When HF and compound of formula (II) are fed separately into DEVICE1-1, then preferably DEVICE1-0 has for each component reagent a respective device: a device DEVICE1-0-HF for conveying the HF, and a device DEVICE1-0-COMP-II for conveying the compound of formula (II).

Preferably, DEVICE1-1 and DEVICE1-2 are during operation in permanent fluid connection with each other and are both under PRESSURE1-1.

Preferably, DEVICE1-0 is the device that builds up PRESSURE1-1 in DEVICE1-1 and in the DEVICE1-2 against the DEVICE1-3, that is necessary to carry out REAC1-1 at TEMP1-1.

More preferably, HF and compound of formula (II) are premixed and then are fed into DEVICE1-1.

PRESSURE1-1 can be the pressure that is needed due to the vapor pressure at the chosen TEMP1-1, PRESSURE1-1 can also be higher than the vapor pressure. Considerations for choosing a PRESSURE1-1 that is higher than the vapor pressure can for example be the requirements of DEVICE1-0. Especially when REAC1-1 is done continuously then PRESSURE1-1 is usually chosen and set to be higher than the vapor pressure.

In case of DEVICE1-1 and any DEVICE1-2 being tubes, especially coiled tubes, due to constructional limitations or due to density fluctuations and the like hot spots or cold spots can occur in spite of efforts to avoid them. Therefore any herein mentioned temperatures are meant to be average temperatures in view of possible hot or cold spots.

Conventional back pressure regulating devices, which can be used for DEVICE1-3, work discontinually, i.e. by alternating opening and closing they release the product stream while holding the pressure. This leads naturally to variations in the pressure. In view of these possible variations of PRESSURE1-1 any pressure mentioned herein is meant to be an average pressure.

All parts in contact with the mixture of HF and compound of formula (II) and with the reaction mixture resulting from REAC1-1 are made out of respective materials which are resistant to the attack of the chemicals under the respective conditions, i.e. stainless steel, hastelloy, such as hastelloy B or hastelloy C, titanium, tantalum, silicon carbide, silicon nitride etc., they can also be passivized or lined with material inert to the chemicals, such as PTFE.

Compound of formula (I) can be used from DEVICE1-3.

Preferably any gaseous components are separated from compound of formula (I). This separation is preferably done in DEVICE1-4. Therefore compound of formula (I) can be used from DEVICE1-3 or from DEVICE1-4 for any subsequent reaction, preferably without further purification. The product from DEVICE1-3 or from DEVICE1-4 can be subjected to a further purification, preferably, the liquid phase obtained from DEVICE1-3 or from DEVICE1-4 is further purified by removing any residual low boiling residues, preferably this is done by using a film evaporator, wiped film evaporator, falling film evaporation, distillation, rectification, flash distillation or short path distillation; more preferably a wiped film evaporator.

Compound of formula (II) is a known compound and can be prepared by known methods.

Compound of formula (II) can be used in purified form, for example purified by distillation or evaporation and any other known methods.

Compound of formula (II), that is reacted in REAC1-1 with HF, can also be used for REAC1-1 in form of a mixture MIX-II-III or in form of a mixture MIXTURE-TRIPLE; thereby compound of formula (III) is present at the beginning of REAC1-1.

MIX-II-III is a mixture of compound of formula (II) with compound of formula (III).

Preferably, the amount of compound of formula (III) in MIX-II-III is at least 0.5%, more preferably at least 0.75%, even more preferably at least 1%, especially at least 2%, more especially at least 3%, even more especially at least 4%, the % are % by weight and are based on the total weight of MIX-II-III.

Preferably, MIX-II-III contains not more than 50%, more preferably not more than 25%, even more preferably not more than 15%, especially not more than 12.5%, more especially not more than 10%, of compound of formula (III), the % are % by weight and are based on the total weight of MIX-II-III.

Any of the lower limits can be combined with any of the upper limits of the amount of compound of formula (III) in MIX-II-III.

Preferably, the total content of the two components in MIX-II-III is of from 50 to 100%, more preferably of form 75 to 100%, even more preferably of from 90 to 100%, especially of from 95 to 100%, more especially of from 97.5 to 100%, even more especially of from 98 to 100%, the % being % by weight based on the total weight of MIX-II-III.

MIXTURE-TRIPLE comprises three components, a compound of formula (II), a compound of formula (III) and compound of formula (IV), in the relative ratio of from
2 to 100% of compound of formula (II),
49 to 0% of compound of formula (III), and
49 to 0% of compound of formula (IV);

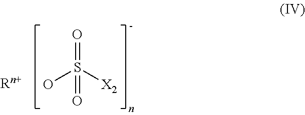

X2 is defined herein, also with all its embodiments;
Rⁿ⁺ is selected from the group consisting of H⁺, Li⁺, Na⁺, K⁺, Mg²⁺, Ca²⁺, Zn²⁺, Cu²⁺, Al³⁺, Ti³⁺, Fe²⁺, Fe³⁺, B³⁺,

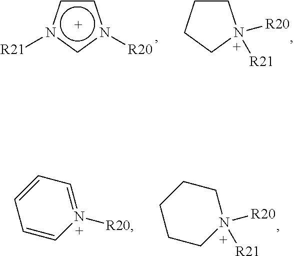

[N(R20)(R21)(R22)R23]⁺, and [P(R20)(R21)(R22)R23]⁺;
R20, R21, R22 and R23 are identical or different and independently from each other selected from the group consisting of H, C₁₋₈ alkyl, C₅₋₆ cycloalkyl, phenyl, benzyl, vinyl and allyl;
n is 1, 2 or 3;
the % are % by weight and are based on the combined weight of said three components in MIXTURE-TRIPLE; the relative ratios of said three components add up to 100%.

The expression “X is identical with X1 or with X2” means that either X stems from compound of formula (III), that means X is X1; or X stems from compound of formula (IV), that means X is X2.

Preferably, in case that X is not F, then X is identical with X2, that means X stems from compound of formula (IV).

Preferably,
Rⁿ⁺ is selected from the group consisting of H⁺, Li⁺, Na⁺, K⁺, Mg²⁺, Ca²⁺, Zn²⁺, Cu²⁺, Al³⁺, Ti³⁺, Fe²⁺, Fe³⁺, B³⁺,

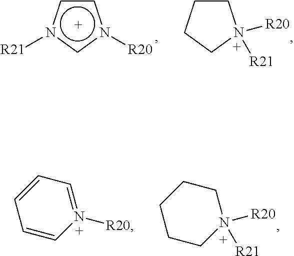

[N(R20)(R21)(R22)R23]⁺, and [P(R20)(R21)(R22)R23]⁺;
R20, R21, R22 and R23 are identical or different and independently from each other selected from the group consisting of H, C₁₋₈ alkyl, C₅₋₆ cycloalkyl, phenyl, benzyl, vinyl and allyl;
n is 1, 2 or 3.

More preferably,
Rⁿ⁺ is selected from the group consisting of H⁺, Li⁺, Na⁺, K⁺, Mg²⁺, Al³⁺,

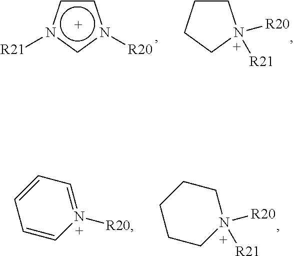

and
[N(R20)(R21)(R22)R23]⁺;
R20, R21, R22 and R23 are identical or different and independently from each other selected from the group consisting of H, C₁₋₄ alkyl, phenyl, benzyl, vinyl and allyl;
n is 1, 2 or 3.

Even more preferably,
Rⁿ⁺ is selected from the group consisting of H⁺, Li⁺, Na⁺,

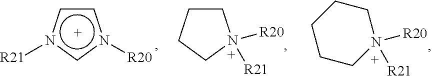

and [N(R20)(R21)(R22)R23]⁺;
R20, R21, R22 and R23 are identical or different and independently from each other selected from the group consisting of H, and C₁₋₄ alkyl;
n is 1.

Especially,
Rⁿ⁺ selected from the group consisting of H⁺, Li⁺, Na⁺,

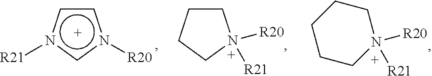

and [N(R20)(R21)(R22)R23]⁺;
R20, R21, R22 and R23 are identical or different and independently from each other C₁₋₄ alkyl;
n is 1.

More especially,
Rⁿ⁺ is selected from the group consisting of H⁺, Li⁺, and Na⁺;
n is 1.

Even more especially,
Rⁿ⁺ is H⁺;
n is 1.

Preferably,
X1 and X2 are identical or different and independently from each other selected from the group consisting of F, Cl, Br, I, RESF, RESF being preferably C₁₋₆ perfluoroalkyl, and tolyl;
Rⁿ⁺ is selected from the group consisting of H⁺, Li⁺, Na⁺, K⁺, Mg²⁺, Ca²⁺, Zn²⁺, Cu²⁺, Al³⁺, Ti³⁺, Fe²⁺, Fe³⁺, B³⁺,

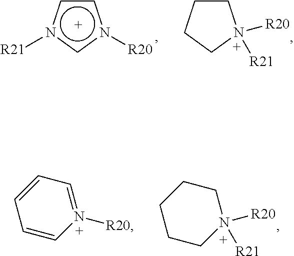

[N(R20)(R21)(R22)R23]⁺, and [P(R20)(R21)(R22)R23]⁺;
R20, R21, R22 and R23 are identical or different and independently from each other selected from the group consisting of H, C₁₋₈ alkyl, C₅₋₆ cycloalkyl, phenyl, benzyl, vinyl and allyl;
n is 1, 2 or 3.

More preferably,
X1 and X2 are identical or different and independently from each other selected from the group consisting of F, Cl, Br, RESF, RESF being preferably C₁₋₆ perfluoroalkyl, and tolyl;
Rⁿ⁺ is selected from the group consisting of H⁺, Li⁺, Na⁺, K⁺, Mg²⁺, Al³⁺,

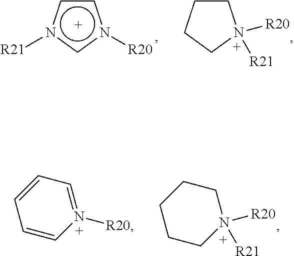

and
[N(R20)(R21)(R22)R23]⁺;
R20, R21, R22 and R23 are identical or different and independently from each other selected from the group consisting of H, C₁₋₄ alkyl, phenyl, benzyl, vinyl and allyl;
n is 1, 2 or 3.

Even more preferably,
X1 and X2 are identical or different and independently from each other selected from the group consisting of F, Cl, and RESF, RESF being preferably C₁₋₄ perfluoroalkyl;
Rⁿ⁺ selected from the group consisting of H⁺, Li⁺, Na⁺,

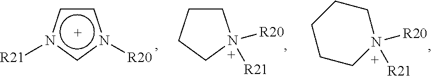

and [N(R20)(R21)(R22)R23]⁺;
R20, R21, R22 and R23 are identical or different and independently from each other selected from the group consisting of H, and C₁₋₄ alkyl;
n is 1.

Especially,
X1 and X2 are identical or different and independently from each other selected from the group consisting of Cl, and RESF, RESF being preferably C₁₋₂ perfluoroalkyl;
Rⁿ⁺ selected from the group consisting of H⁺, Li⁺, Na⁺,

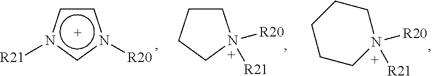

and [N(R20)(R21)(R22)R23]⁺;
R20, R21, R22 and R23 are identical or different and independently from each other C₁₋₄ alkyl;
n is 1.

More especially,
X1 and X2 are identical or different and independently from each Cl or CF₃;
Rⁿ⁺ is selected from the group consisting of H⁺, Li⁺, and Na⁺;
n is 1.

Even more especially,
X1 and X2 are identical or different and independently from each Cl or CF₃;
Rⁿ⁺ is H⁺;
n is 1.

Specific embodiments of compound of formula (IV) are chlorosulfonic acid and trifluoromethyl sulfonic acid.

In one particular embodiment,
compound of formula (I) is compound of formula (1),
compound of formula (II) is compound of formula (2),
compound of formula (III) is compound of formula (3),
and compound of formula (IV) is chlorosulfonic acid,
that means X, X1 and X2 are Cl, Rⁿ⁺ is H⁺ and n is 1.

In another particular embodiment,
compound of formula (I) is compound of formula (1-CF₃),
compound of formula (II) is compound of formula (2-CF₃),
compound of formula (III) is compound of formula (3),
and compound of formula (IV) is trifluoromethyl sulfonic acid,
that means X1 is Cl, Rⁿ⁺ is H⁺, and X and X2 are CF₃, and n is 1.

In a preferred embodiment, MIXTURE-TRIPLE comprises the three components, compound of formula (II), compound of formula (III) and compound of formula (IV).

In another preferred embodiment, MIXTURE-TRIPLE comprises compound of formula (II), but does not comprise compound of formula (III) or compound of formula (IV), at least not in essential amounts. This is for example the case if compound of formula (III) is used in purified form, for example purified by distillation or evaporation and the like.

In another preferred embodiment, MIXTURE-TRIPLE consists essentially of compound of formula (II).

In another preferred embodiment, MIXTURE-TRIPLE consists essentially of the three components compound of formula (II), compound of formula (III), and compound of formula (IV).

In another preferred embodiment, the relative ratio of the three components in MIXTURE-TRIPLE is of from
2 to 100% of compound of formula (II),
49 to 0% of compound of formula (III), and
49 to 0% of compound of formula (IV);
   more preferably of from
2 to 99% of compound of formula (II),
49 to 0.5% of compound of formula (III), and
49 to 0.5% of compound of formula (IV);
   even more preferably of from
2 to 98% of compound of formula (II),
49 to 1% of compound of formula (III), and
49 to 1% of compound of formula (IV);
   especially of from
2 to 96% of compound of formula (II),
49 to 2% of compound of formula (III), and
49 to 2% of compound of formula (IV);
   more especially of from
50 to 96% of compound of formula (II),
25 to 2% of compound of formula (III), and
25 to 2% of compound of formula (IV);
   even more especially of from
70 to 96% of compound of formula (II),
15 to 2% of compound of formula (III), and
15 to 2% of compound of formula (IV);
   in particular of from
75 to 96% of compound of formula (II),
12.5 to 2% of compound of formula (III), and
12.5 to 2% of compound of formula (IV);
   more in particular of from
75 to 94% of compound of formula (II),
12.5 to 3% of compound of formula (III), and
12.5 to 3% of compound of formula (IV);
   even more in particular of from
75 to 92% of compound of formula (II),
12.5 to 4% of compound of formula (III), and
12.5 to 4% of compound of formula (IV);
   the % are % by weight and are based on the combined weight of the three components in MIXTURE-TRIPLE; the relative ratios of the three components add up to 100%.

Preferably, the total content of the three components in MIXTURE-TRIPLE is of from 50 to 100%, more preferably of form 75 to 100%, even more preferably of from 90 to 100%, especially of from 95 to 100%, more especially of from 97.5 to 100%, even more especially of from 98 to 100%, the % being % by weight based on the total weight of MIXTURE-TRIPLE.

Preferably, compound of formula (II), that is reacted in REAC1-1 with HF, is used for REAC1-1 in form of MIX-II-III or in form of MIXTURE-TRIPLE.

Preferably, compound of formula (II), MIX-II-III or MIXTURE-TRIPLE is prepared in a step STEP0;
STEP0 comprises a reaction REAC0-1;
REAC0-1 is a reaction of compound of formula (III) with compound of formula (IV).

STEP0 is done before STEP1.

Preferably, the molar amount of compound of formula (IV) in REAC0-1 is from 0.5 to 1.5 fold, more preferably from 0.75 to 1.25 fold, even more preferably from 0.85 to 1.15 fold, of the molar amount of compound of formula (III).

Preferably, REAC0-1 is done at a temperature TEMP0-1, TEMP0-1 is from 180 to 300° C., more preferably from 190 to 280° C., even more preferably from 200 to 260° C., especially from 210 to 255° C., more especially from 220 to 255° C.

Preferably, REAC0-1 is done in a time TIME0-1, TIME0-1 is from 0.5 sec to 4 h, more preferably from 1 sec to 2 h, even more preferably 1 min to 1 h, especially from 2 min to 30 min, more especially from 2 min to 20 min, even more especially from 3 min to 17 min.

REAC0-1 is done at a pressure PRESSURE0-1, preferably, PRESSURE0-1 is from 10 to 1000 bar, more preferably from 20 to 600 bar, even more preferably from 50 to 500 bar, especially from 60 to 400 bar, more especially from 65 to 300 bar, even more from 65 to 200 bar, in particular from 65 to 150 bar.

Preferably, REAC0-1 is done in a continuous way.

In a preferred embodiment, STEP0 comprises one step, the step STEP0-1;
STEP0-1 comprises the reaction REAC0-1;
in STEP0-1 a mixture MIXTURE0-1 of compound of formula (III) and compound of formula (IV) is heated in DEVICE0-1 to TEMP0-1, REAC0-1 takes place in DEVICE0-1 resulting in a reaction mixture.

In another more preferred embodiment, STEP0 comprises another step STEP0-3;
STEP0-3 is done after STEP0-1;
in STEP0-3 the reaction mixture from DEVICE0-1 passes through a device DEVICE0-3, DEVICE0-3 is a device for back pressure regulation.

In another more preferred embodiment, STEP0 comprises another step STEP0-2;
STEP0-2 is done after STEP0-1 or after STEP0-3;
in STEP0-2 the reaction mixture from DEVICE0-1 or from DEVICE0-3 passes through a device DEVICE0-2, DEVICE0-2 is a device for cooling the reaction mixture;

In another preferred embodiment, STEP0 comprises all three steps STEP0-1, STEP0-2 and STEP0-3;

preferably, STEP0-2 is done after STEP0-1 and before STEP03.

Preferably, the reaction mixture is cooled by the effects of DEVICE0-2 or of DEVICE0-3 or of a combination of DEVICE0-2 and DEVICE0-3 on the reaction mixture.

Preferably, DEVICE0-1, DEVICE0-2 and DEVICE0-3 are continuously working devices.

Preferably, the method comprises another step STEP0-4, which is done after STEP0-3, in STEP0-4 the reaction mixture from DEVICE0-3 passes through a device DEVICE0-4, DEVICE0-4 is a device for separating CO₂ from the reaction mixture.

Preferably, the REAC0-1 is done in a tubular reactor.

Preferably, MIXTURE0-1 is fed into DEVICE0-1, during the passage through DEVICE0-1, the initially fed MIXTURE0-1 gradually is converted to the reaction mixture by REAC0-1.

The reaction mixture from DEVICE0-1, DEVICE0-2, DEVICE0-3 or DEVICE0-4 can be compound of formula (II), MIX-II-III or MIXTURE-TRIPLE.

MIX-II-III and MIXTURE-TRIPLE can be prepared in according to known methods, for example by mixing the three components.

Other components in MIX-II-III or in MIXTURE-TRIPLE besides the respective two or three components can be e.g. a solvent, REAC0-1 can be done in the presence of such a solvent. Such a solvent can be any solvent that is inert against the two or three components of MIX-II-III and MIXTURE-TRIPLE respectively and preferably also against HF. Examples for such solvents are disclosed in US 2015/0246812 A1.

As organic solvent, mention may in particular be made of esters, nitriles or dinitriles, ethers or diethers, amines or phosphines, such as for example methyl acetate, ethyl acetate, butyl acetate, acetonitrile, propionitrile, isobutyronitrile, glutaronitrile, dioxane, tetrahydrofuran, methyl tetrahydrofuran, triethylamine, tripropylamine, diethylisopropylamine, pyridine, trimethylphosphine, triethylphosphine and diethylisopropylphosphine, preferably ethyl acetate, butyl acetate, acetonitrile, dioxane, tetrahydrofuran and methyl tetrahydrofuran.

Also REAC1-1 can be done in the presence of such a solvent.

Preferably, REAC0-1 or REAC1-1 or both are done in the absence of an organic solvent.

Preferably, REAC0-1 or REAC1-1 or both are done in the absence of an organic base containing nitrogen.

Preferably, REAC0-1 or REAC1-1 or both are done in the absence of a salt of an organic base containing nitrogen.

Preferably, REAC0-1 or REAC1-1 or both are done in the absence of an organic base.

Preferably, REAC0-1 or REAC1-1 or both are done in the absence of a salt of an organic base.

Preferably, REAC0-1 or REAC1-1 or both are done in the absence of a base.

Preferably, REAC0-1 or REAC1-1 or both are done in the absence of a salt of a base.

Preferably, REAC0-1 or REAC1-1 or both are done in the absence of a salt of a base.

Preferably, REAC0-1 or REAC1-1 or both are done in the absence of a metal salt.

Preferably, the reaction mixture from DEVICE0-1, DEVICE0-2, DEVICE0-3 or DEVICE0-4 is MIX-II-III or MIXTURE-TRIPLE.

Preferably, DEVICE0-1 is selected from the group consisting of tube, microreactor, shell and tube heat exchanger, plate heat exchanger and any common device which purpose is to exchange heat from a mixture;

more preferably it is a tube;
even more preferably it is a coiled tube.

Preferably, DEVICE0-2 is selected from the group consisting of tube, microreactor, shell and tube heat exchanger, plate heat exchanger and any common device which purpose is to exchange heat from a reaction mixture;

more preferably it is a tube;
even more preferably it is a coiled tube.

Especially, DEVICE0-1 and DEVICE0-2 are coiled tubes.

Preferably, DEVICE0-3 is a conventional back pressure regulating device.

Preferably, DEVICE0-4 a device capable of separating gaseous CO₂ from a liquid, any known device suitable for this purpose for can be used for this purpose, more preferably DEVICE0-4 is a column, a cyclone or a vessel.

The heating, preferably in DEVICE0-1, can be done be any known means, preferably it is done by electric heating or by heating with a fluid heat carrier.

Cooling in DEVICE0-2 can be done be any known means, preferably it is done by a fluid cooling medium.

Depending on the scale of the reaction and thereby on the scale of the apparatus, wherein the method is done, the cooling of the reaction mixture can be done by the effect of DEVICE0-2 on the reaction mixture, i.e. during the passage of the reaction mixture through DEVICE0-2, or it can be done by the effects of DEVICE0-3 on the reaction mixture, i.e. the passage through DEVICE0-3, contributes to the cooling. This is especially the case when the scale of the reaction is rather small, e.g. when the method is done on lab scale, whereas in case where the method is done on a production scale the cooling will usually primarily be done during the passage through DEVICE0-2.
In another embodiment, especially on production scale, cooling can also be achieved by the expansion and pressure release affected by DEVICE0-3.
Also a combination of cooling during the passage through DEVICE0-2 with a cooling by expansion affected by DEVICE0-3 is possible.

Preferably, heating in DEVICE0-1 and cooling in DEVICE0-2 is realized in form of a tube-in-tube set up, in form of a tube-in-container set up, in form of a shell and tube heat exchanger, plate heat exchanger or any common device which purpose is to exchange heat from a mixture or a reaction mixture;
more preferably, heating in DEVICE0-1 and cooling in DEVICE0-2 is realized in form of a tube-in-tube set up or in form of a tube-in-container set up.

REAC0-1 is triggered, preferably in DEVICE0-1, by the heating of MIXTURE0-1 to TEMP0-1.

The cooling in STEP0-2 is preferably done to a temperature TEMP0-2, preferably TEMP0-2 is from 0 to 180° C., more preferably from 0 to 150° C., even more preferably from 10 to 120° C., especially from 15 to 100° C., more especially from 15 to 90° C., even more especially from 15 to 85° C., in particular from 20 to 85° C.

Preferably, REAC0-1 is quenched by the cooling of the reaction mixture in DEVICE0-2 or in DEVICE0-3 or in both, preferably by cooling to TEMP0-2.

When compound of formula (2) is prepared in REAC0-1 by reaction of compound of formula (3) with chlorosulfonic acid, then the melting point of pure compound of formula (2) is ca. 35° C., therefore the lowest possible value of TEMP0-2 is governed by the conversion of the reaction, since residual compound of formula (3) and residual chlorosulfonic acid in the reaction mixture naturally lowers the melting point of the reaction mixture after the reaction and allows for lower values of TEMP0-2.

PRESSURE0-1 in DEVICE0-1 and in optional DEVICE0-2 is controlled and held by the DEVICE0-3.

TIME0-1 is the time, where MIXTURE0-1 is exposed to heating and to the TEMP0-1. During TIME0-1 the REAC0-1 takes place. Preferably TIME0-1 is therefore a residence time and when REAC0-1 takes place in DEVICE0-1, then TIME0-1 is preferably the residence time of the mixture in DEVICE0-1.

Time TIME0-2 is the time, where the reaction mixture is cooled to TEMP0-2. The cooling can be done by the action of DEVICE0-2, by the action of DEVICE0-3 or by the action of DEVICE0-2 and DEVICE0-3. The cooling quenches the reaction. Preferably TIME0-2 is therefore a residence time and is preferably the residence time of the reaction mixture in DEVICE0-2, in DEVICE0-3 or in both.

Preferably, TIME0-2 is from 0.1 sec to 2 h, more preferably from 0.5 sec to 1 h, even more preferably 1 sec to 30 min, especially from 10 sec to 30 min, more especially from 25 sec to 25 min, even more especially from 1 min to 25 min.

Preferably, TIME0-2 is from 0.0001 to 0.5 fold of time, more preferably from 0.001 to 0.3 fold, of TIMED-1.

Compound of formula (III) and compound of formula (IV) can be fed into the DEVICE0-1 as a premixed mixture or can be fed into the DEVICE0-1 separately and are mixed in DEVICE0-1.

For the purpose of mixing before or in DEVICE0-1 any suitable installation for mixing can be used, which are known in the state of the art, such as a common branch connection, e.g. a T or Y piece, or a static mixing device.

Preferably, the heating to TEMP0-1 in DEVICE0-1 is done only after compound of formula (III) and compound of formula (IV) are present as a mixture in DEVICE0-1.

The feeding of compound of formula (III) and compound of formula (IV), either separately or in form of a mixture, is done by a device DEVICE0-0.

DEVICE0-0 is a pressuring device conventionally used to convey a fluid against pressure, such as a pump. When compound of formula (III) and compound of formula (IV) are fed separately into DEVICE0-1, then preferably DEVICE0-0 has for each component a respective device, a device DEVICE0-0-COMP3 for conveying the compound of formula (III), and a device DEVICE0-0-CSA for conveying the compound of formula (IV).

Preferably, DEVICE0-1 and any DEVICE0-2 and any DEVICE0-3 are during operation in permanent fluid connection with each other and are both under PRESSURE0-1.

Preferably, DEVICE0-0 is the device that builds up the PRESSURE0-1 in DEVICE0-1 and in the DEVICE0-2 against the DEVICE0-3, which is necessary to carry out the REAC0-1 at the TEMP0-1.

More preferably, compound of formula (III) and compound of formula (IV) are mixed under ambient pressure and at ambient temperature and then are fed into DEVICE0-1.

In case of DEVICE0-1 and/or DEVICE0-2 being tubes, especially coiled tubes, due to constructional limitations or due to density fluctuations and the like hot spots or cold spots can occur in spite of efforts to avoid them. Therefore any mentioned temperatures are meant to be average temperatures in view of possible hot or cold spots.

Conventional back pressure regulating devices, which can be used for DEVICE0-3, work usually discontinually, i.e. by opening and closing they release the product stream while holding the pressure. This leads naturally to variations in the pressure. Therefore the PRESSURE0-1 is meant to be an average pressure.

All parts in contact with MIXTURE0-1 and with the reaction mixture are made out of respective materials, which are resistant to the attack of the chemicals under the respective conditions, i.e. stainless steel, hastelloy, such as hastelloy B or hastelloy C, titanium, tantalum, silicon carbide, silicon nitride etc., they can also be passivized or lined with material inert to the chemicals, such as PTFE.

Compound of formula (II), MIX-II-III or MIXTURE-TRIPLE can be used from DEVICE0-1, from DEVICE0-2, from DEVICE0-3 or from DEVICE0-4, preferably from DEVICE0-3 or from DEVICE0-4, for REAC1-1 without further purification, in case of a further purification, preferably, compound of formula (II), MIX-II-III or MIXTURE-TRIPLE, such as the liquid phase obtained from DEVICE0-4, is further purified by removing any low boiling residues, preferably this is done by using a film evaporator, wiped film evaporator, falling film evaporation, distillation, rectification, flash distillation or short path distillation; more preferably a wiped film evaporator.

In an especially preferred embodiment, REAC0-1 and REAC1-1 are done continuously and consecutively, preferably without interruption of the flow of the components; preferably DEVICE0-1 and DEVICE1-1 are connected, preferably in fluid connection, for example via DEVICE0-2.

In this case, DEVICE0-3, DEVICE0-4 and DEVICE1-0 are not mandatorily required, rather PRESSURE0-1 and PRESSURE1-1 can be identical and can be build up by DEVICE0-0 against the action of DEVICE1-3.

Depending on the dimensions and the construction of the whole apparatus setup, also DEVICE0-2 is not mandatorily required, or DEVICE0-2 can simply be realized by the device or devices, such as tubes, which connect DEVICE0-1 and DEVICE1-1.

Preferably, the reaction mixture from DEVICE0-1 or from any DEVICE0-2 can be used as compound of formula (II), MIX-II-III or MIXTURE-TRIPLE for REAC1-1;
more preferably, the reaction mixture from any DEVICE0-2 can be used as compound of formula (II), MIX-II-III or MIXTURE-TRIPLE for REAC1-1;
even more preferably, the reaction mixture from DEVICE0-1 is cooled in DEVICE0-2 to a temperature TEMP0-2 of from 120 to 210° C., preferably of from 120 to 200° C., more preferably of from 120 to 180° C.; and then the mixture from DEVICE0-2 is used as compound of formula (II), MIX-II-III or MIXTURE-TRIPLE for REAC1-1 in DEVICE1-1.

In another preferred embodiment, the reaction mixture from any DEVICE0-3 or from any DEVICE0-4 can be used as compound of formula (II), MIX-II-III or MIXTURE-TRIPLE for REAC1-1.

In another preferred embodiment, PRESSURE0-1 and PRESSURE1-1 are not identical, more preferably PRESSURE1-1 is lower than PRESSURE0-1;
more preferably the reaction mixture from a DEVICE0-3 is used as compound of formula (II), MIX-II-III or MIXTURE-TRIPLE for REAC1-1;
even more preferably the reaction mixture from a DEVICE0-4 is used as compound of formula (II), MIX-II-III or MIXTURE-TRIPLE for REAC1-1.

In case that the reaction mixture from DEVICE0-1 or from DEVICE0-2 is used directly as compound of formula (II), MIX-II-III or MIXTURE-TRIPLE for REAC1-1, any cooling after STEP0-1, preferably the cooling in STEP0-2, does not have to be as intensive as in case that the reaction mixture from REAC0-1, that is the reaction mixture from DEVICE0-1, DEVICE0-2 or from DEVICE0-3, is not used directly and immediately as compound of formula (II), MIX-II-III or MIXTURE-TRIPLE for REAC1-1, but there is some time interval in between. In this case any cooling after STEP0-1, such as the cooling in STEP0-2, should preferably ensure that the target temperature after such cooling is below the decomposition temperature of the reaction mixture obtained from REAC0-1.

Preferably, MIX is done using compound of formula (I) as obtained from DEVICE1-3 or from DEVICE1-4 to provide MIXWAT.

MIX is done batch wise or in a continuous way;
preferably, MIX is done continuously;
more preferably, REAC1-1 and MIX are done continuously;
even more preferably, REAC1-1 and MIX are done continuously and consecutively;
especially, REAC1-1 and MIX are done continuously and consecutively without interruption of the flow of the components.

EXTR is done batch wise or in a continuous way;
preferably, EXTR is done continuously;
more preferably, REAC1-1, MIX and EXTR are done continuously;
even more preferably, REAC1-1, MIX and EXTR are done continuously and consecutively;
especially, REAC1-1, MIX and EXTR are done continuously and consecutively without interruption of the flow of the components.

REAC2 is done batch wise or in a continuous way;
preferably, REAC2 is done continuously;
more preferably, REAC1-1, MIX, EXTR and REAC2 are done continuously;
even more preferably, REAC1-1, MIX, EXTR and REAC2 are done continuously and consecutively;
especially, REAC1-1, MIX, EXTR and REAC2 are done continuously and consecutively without interruption of the flow of the components.

In particular, REAC0-1, REAC1-1 and MIX are done continuously;
preferably, REAC0-1, REAC1-1 and MIX are done continuously and consecutively;
more preferably, REAC0-1, REAC1-1 and MIX are done continuously and consecutively without interruption of the flow of the components.

In another particular embodiment, REAC0-1, REAC1-1, MIX and EXTR are done continuously;
preferably, REAC0-1, REAC1-1, MIX and EXTR are done continuously and consecutively;
more preferably, REAC0-1, REAC1-1, MIX and EXTR are done continuously and consecutively without interruption of the flow of the components.

In another particular embodiment, REAC0-1, REAC1-1, MIX, EXTR and REAC2 are done continuously;
preferably, REAC0-1, REAC1-1, MIX, EXTR and REAC2 are done continuously and consecutively;
more preferably, REAC0-1, REAC1-1, MIX, EXTR and REAC2 are done continuously and consecutively without interruption of the flow of the components.

In another particular embodiment, REAC1-1 and REAC2 are done continuously; preferably, REAC0-1, REAC1-1 and REAC2 are done continuously;
more preferably, REAC0-1, REAC1-1, and REAC2 are done continuously and without interruption of the flow of the components.

REAC2-1 is done batch wise or in a continuous way;
preferably, REAC2-1 is done continuously;
more preferably, REAC1-1 and REAC2-1 are done continuously;
even more preferably, REAC1-1 and REAC2-1 are done continuously and consecutively;
especially, REAC1-1 and REAC2-1 are done continuously and consecutively without interruption of the flow of the components.

EXTR2-1 is done batch wise or in a continuous way;
preferably, EXTR2-1 is done continuously;
more preferably, REAC1-1, REAC2-1 and EXTR2-1 are done continuously;
even more preferably, REAC1-1, REAC2-1 and EXTR2-1 are done continuously and consecutively;
especially, REAC1-1, REAC2-1 and EXTR2-1 are done continuously and consecutively without interruption of the flow of the components.

REAC2-2 is done batch wise or in a continuous way;
preferably, REAC2-2 is done continuously;
more preferably, REAC1-1, REAC2-1, EXTR2-1 and REAC2-2 are done continuously;
even more preferably, REAC1-1, REAC2-1, EXTR2-1 and REAC2-2 are done continuously and consecutively;
especially, REAC1-1, REAC2-1, EXTR2-1 and REAC2-2 are done continuously and consecutively without interruption of the flow of the components.

In particular, REAC0-1, REAC1-1 and REAC2-1 are done continuously;
preferably, REAC0-1, REAC1-1 and REAC2-1 are done continuously and consecutively;
more preferably, REAC0-1, REAC1-1 and REAC2-1 are done continuously and consecutively without interruption of the flow of the components.

In another particular embodiment, REAC0-1, REAC1-1, REAC2-1 and EXTR2-1 are done continuously;
preferably, REAC0-1, REAC1-1, REAC2-1 and EXTR2-1 are done continuously and consecutively;
more preferably, REAC0-1, REAC1-1, REAC2-1 and EXTR2-1 are done continuously and consecutively without interruption of the flow of the components.

In another particular embodiment, REAC0-1, REAC1-1, REAC2-1, EXTR2-1 and REAC2-2 are done continuously;
preferably, REAC0-1, REAC1-1, REAC2-1, EXTR2-1 and REAC2-2 are done continuously and consecutively;
more preferably, REAC0-1, REAC1-1, REAC2-1, EXTR2-1 and REAC2-2 are done continuously and consecutively without interruption of the flow of the components.

Each of the described steps is preferably done continuously, and any of the described steps in any of the described combinations are preferably done continuously and without interruption of the flow of the components.

### EXAMPLES

### Methods:

The yield of HFSI was determined by ¹⁹F-NMR using benzenesulfonylfluoride as internal reference in D₃-acetonitrile as solvent, if not otherwise stated

In general, if not otherwise stated, the products containing derivatives of FSI, such as LiFSI, HFSI, NaFSI and the like, were analyzed and characterized at least with ¹⁹F NMR, sometimes also elementary analysis, e.g. for determination of a respective metal, GC (gas chromatography), e.g. for analyzing solvents, and ion chromatography, e.g. for detection of ionic impurities, were used.

### Materials

Compound of formula (II) is prepared according to example 15 of WO 2015/004220 A1. The conversion of 95% stated in this example 15 of WO 2015/004220 A1 means that 5% residual CSI are present in this compound of formula (II). Thereby this compound of formula (II) can be seen as a MIX-II-III containing 5% of residual CSI, a MIX-II-III-5. It is assumed that therefore the equivalent amount of chlorosulfonic acid is present in this compound of formula (II) as well. Thereby this compound of formula (II) can also be seen as a mixture MIXTURE-TRIPLE-90-5-5; MIXTURE-TRIPLE-90-5-5 contains ca. 90% of compound of formula (2), 5% of compound of formula (3) and 5% of chlorosulfonic acid, the % being % by weight based on the total weight of MIXTURE-TRIPLE-90-5-5. In the following “MIXTURE-TRIPLE-90-5-5” means said compound of formula (II) and said MIX-II-III-5.

Compound of formula (II) is prepared according to example 5 of WO 2015/004220 A1. The conversion of 92.4% stated in this example 5 of WO 2015/004220 A1 means that 7.6% of residual CSI are present in this compound of formula (II). Thereby this compound of formula (II) can be seen as a MIX-II-III containing roughly 7.5% of residual CIS, a MIX-II-III-7.5. It is assumed that therefore the equivalent amount of chlorosulfonic acid is present in this compound of formula (II) as well. Thereby this compound of formula (II) can also be seen as a mixture MIXTURE-TRIPLE-85-7.5-7.5; MIXTURE-TRIPLE-85-7.5-7.5 contains roughly 85% of compound of formula (2), roughly 7.5% of compound of formula (3) and roughly 7.5% of chlorosulfonic acid, the % being % by weight based on the total weight of MIXTURE-TRIPLE-85-7.5-7.5. In the following “MIXTURE-TRIPLE-85-7.5-7.5” means said compound of formula (II) and said MIX-II-III-7.5.

### Example 1

The examples were carried out with
DEVICE1-0-HF: piston pump 260D from ISCO Teledyne
DEVICE1-0-COMP-II: piston pump 260D from ISCO Teledyne
DEVICE1-1 being a ⅛ inch coiled tube with internal volume VOLUME1 of 3.8 ml made of hastelloy C. For the heating a coiled-tube-in-container set up was used. Heating medium was conventional oil.
DEVICE1-2 being a ⅛ inch tube with ca. 1.5 mL internal volume made of hastelloy C. Cooling was done by simply contact of the tube with the air having room temperature.
DEVICE1-3: pneumatically controlled valve from SAMSON Microvalve type 3510-7 with a Cv value of 0.01.
DEVICE1-4: any gaseous components, which are essentially HCl and excess HF, were separated from the reaction mixture in a vented vessel made of stainless steel.

MIXTURE-TRIPLE-90-5-5 was fed simultaneously with HF into DEVICE1-1 at a PRESSURE1-1 of 80 bar, MIXTURE-TRIPLE-90-5-5 was fed by DEVICE1-0-COMP-II at a flow rate of 0.118 ml/min, and HF was fed by DEVICE1-0-HF with at a flow rate of 0.137 ml/min. TIME1-1 was approximately 15 min, TEMP1-1 was 160° C. The molar ratio of HF:MIXTURE-TRIPLE-90-5-5 resulting from the flow rates was approximately 8:1. Then the reaction mixture from DEVICE1-1 was cooled to TEMP1-2 in DEVICE1-2, TEMP1-2 was room temperature, TIME1-2 was approximately 5.9 min, and was then expanded by DEVICE1-3 into DEVICE1-4. The liquid collected was HFSI confirmed by ¹⁹F NMR.

The yield was 89% based on compound of formula (2) in MIXTURE-TRIPLE-90-5-5.

### Example 2

Example 1 was repeated with the sole difference, that MIXTURE-TRIPLE-90-5-5 was fed by DEVICE1-O-COMP-II at a flow rate of 0.198 ml/min, and HF was fed by DEVICE1-0-HF with at a flow rate of 0.057 ml/min, resulting in a molar ratio of HF:MIXTURE-TRIPLE-90-5-5 from the flow rates of approximately 2:1.

The other parameters were the same as in example 1.

The yield was 72% based on compound of formula (2) in MIXTURE-TRIPLE-90-5-5.

### Example 3

The example was carried out with
DEVICE0-0: piston pump 260D from ISCO Teledyne
DEVICE1-0-HF: piston pump 260D from ISCO Teledyne
DEVICE0-1: a ⅛ inch coiled tube with internal volume VOLUME0 of 5 ml made of hastelloy C. For the heating a coiled-tube-in-container set up was used. Heating medium was conventional oil.
DEVICE1-1 being a ⅛ inch coiled tube with internal volume VOLUME1 of 3.8 ml made of hastelloy B. For the heating a coiled-tube-in-container set up was used. Heating medium was conventional oil.
DEVICE1-2 being a ⅛ inch tube with ca. 1.5 mL internal volume made of hastelloy C. Cooling was done by simply contact of the tube with the air having room temperature.
DEVICE1-3: pneumatically controlled valve from SAMSON Microvalve type 3510-7 with a Cv value of 0.01.
DEVICE1-4: a glass vessel filled with aqueous NaOH 15 wt % for quenching purpose and for neutralizing any HCl and HF.

An equimolar mixture of CSOS and compound of formula (3) was fed by DEVICE0-0 into DEVICE0-1 at a PRESSURE0-1 of 80 bar and with a flow rate of 0.77 ml/min. TEMP0-1 of DEVICE0-1 was 230° C., TIME0-1 was approximately 5 min.

A stream of the resulting MIXTURE-TRIPLE of this example left DEVICE0-1.

A sample was taken of this MIXTURE-TRIPLE, analysis revealed a content of approximately 10.7 wt % of compound of formula (3).

This MIXTURE-TRIPLE therefore is a compound of formula (II) containing 10.7% of residual CSI are present in this compound of formula (II). Thereby this compound of formula (II) can be seen as a MIX-II-III containing 10.7% of residual CIS, a MIX-II-III-10. It is assumed that the equivalent amount of chlorosulfonic acid is present in this compound of formula (II) as well, which means a relative ratio of the three components in this MIXTURE-TRIPLE of approximately
80% of compound of formula (2),
10% of compound of formula (3), and
10% of chlorosulfonic acid;
   the % being % by weight based on the total weight of this MIXTURE-TRIPLE.

Thereby this compound of formula (II) can also be seen as a mixture MIXTURE-TRIPLE-80-10-10; MIXTURE-TRIPLE-80-10-10 contains roughly 80% of compound of formula (2), roughly 10% of compound of formula (3) and roughly 10% of chlorosulfonic acid, the % being % by weight based on the total weight of MIXTURE-TRIPLE-80-10-10. In the following “MIXTURE-TRIPLE-80-10-10” means said compound of formula (II) and said MIX-II-III-10. Then HF with room temperature was fed at PRESSURE1-1 of 80 bar with a flow rate of 0.24 ml/min by DEVICE1-0-HF into this stream of this MIXTURE-TRIPLE, resulting in a mixture of this MIXTURE-TRIPLE and HF, which entered DEVICE 1-1. TEMP1-1 of DEVICE1-1 was 160° C., TIME1-1 was approximately 3 min. The molar ratio of HF:this MIXTURE-TRIPLE resulting from the flow rates was approximately 3:1. The reaction mixture leaving DEVICE1-1 then entered into DEVICE1-2, TEMP1-2 was room temperature. The reaction mixture leaving DEVICE1-2 was then expanded by DEVICE1-3 and then was fed into DEVICE1-4 for quenching purpose. A sample of the reaction mixture was taken between DEVICE1-3 and DEVICE1-4, the sample was mixed with water (1 part by weight of sample with 9 parts by weight of water) and analyzed by ¹⁹F NMR which confirmed that it was HFSI.

The yield was 70% based on compound of formula (3).

### Example 4

Example 1 was repeated with the differences:

MIXTURE-TRIPLE-90-5-5 was fed by DEVICE1-O-COMP-II at a flow rate of 1.07 ml/min.

HF was fed by DEVICE1-0-HF with at a flow rate of 0.46 ml/min.

TIME1-1 was approximately 2.5 min.

The molar ratio of HF:MIXTURE-TRIPLE-90-5-5 resulting from the flow rates was approximately 3:1.

TIME1-2 was approximately 1.5 min.

The yield was 90% based on compound of formula (2) in MIXTURE-TRIPLE-90-5-5.

### Example 5

Example 1 was repeated with the differences:

MIXTURE-TRIPLE-85-7.5-7.5 was fed by DEVICE1-0-COMP-II at a flow rate of 1.22 g/min.

HF was fed by DEVICE1-0-HF with at a flow rate of 0.18 g/min.

TIME1-1 was approximately 5 min.

The molar ratio of HF:MIXTURE-TRIPLE-85-7.5-7.5 resulting from the flow rates was approximately 1.9:1.

Under these conditions HFSI was produced.

Yield 61.6%

### Example 6

234.9 g HFSI, prepared according to example 5, was added to a solution of water (373 g) and TEA (285.2 g) while maintaining a temperature of 10 to 20° C. Then the pH value was adjusted to 9 by addition of TEA (125.6 g). Then the mixture was extracted with VN (2 times with 140 g each). The organic layers were combined (475.68 g, 21.69 wt % HFSI, determined by quantitative ¹⁹F-NMR in ACN) and were extracted with water (2 times with 153.6 g each) at 25° C. NH₃ (3.91 g) was added to the organic layer whereby again an aqueous layer was formed which was separated and discarded. Then aqueous LiOH (95.81 g, ca. 12.5 wt %, prepared from LiOH x H₂O “battery grade” and water) was added to the organic layer, the aqueous layer that was formed was separated and discarded and then aqueous LiOH (95.61 g, ca. 12.5 wt %, prepared from LiOH x H₂O “battery grade” and water) was added. The aqueous layer was again separated and discarded. Obtained was a solution of LiFSI in VN/TEA (414.83 g).

The solution was concentrated under vacuo (30 mbar) at 60° C. and filtered to provide a solution of LiFSI (140.22 g, 36.71 wt %). Then this solution of LiFSI was distilled under vacuo (ca. 7 mbar) at 60° C. During the distillation DCB (543 g) was continuously added and at the same time distillate (467 g) was collected, while maintaining approximately always the same volume in the distillation vessel. After the addition of DCB was completed, crystals had formed and were collected by filtration and washed with DCM (2 times with 50 g each). The crystals were dried under vacuo at 60° C. LiFSI (40.23 g) was obtained as white solid.

### Example 7

1059.4 g of an aqueous solution of HFSI (5% by weight of HFSI based on the total weight of the aqueous solution; prepared according to example 3 by taking the reaction mixture between DEVICE1-3 and DEVICE1-4 and then mixing the reaction mixture with the respective amount of water) were charged into a PTFE reactor, then 169.8 g of methyl-tert butyl ether (MTBE) were added and mixed at 25° C., then the layers which were formed were separated. The extraction was repeated once. The combined organic layers from the two extractions (368.4 g) were charged back to the reactor and was cooled to ca. −2° C., and then 120.5 g of an aqueous solution of LiOH monohydrate (17% by weight of LiOH monohydrate) were added at a temperature of from ca. −2° C. to ca. 20° C. 64.7 g of methyl-tert butyl ether were added. Two layers had formed, the layers were separated. MTBE was removed in vacuum at 60° C. until a concentration of around 40% by weight of LiFSI in MTBE was achieved. LiFSI was crystallized by further evaporation of MTBE under reduced pressure (from 200 mbar to 5 mbar) and at a temperature from 12° C. to 55° C., during this evaporation of MTBE the LiFSI crystallized and formed a suspension, and simultaneously with this evaporation of MTBE, 715 g of 1,2 dichlorobenzene as antisolvent were dosed at such a rate so to keep to keep the volume of the suspension approximately constant. The suspension was filtered and the filter cake was washed (three times with ca. 70 g dichloromethane each time), LiFSI was isolated as white crystals. After drying of the crystals at 60° C. for 3 h, 20.6 g of LiFSI was isolated as white powder.

### Comparative Example (i)

Of example 10 of U.S. Pat. No. 7,919,629 B2, the experiment with 2 h at 130° C. was repeated.

The yield was 55%, which is the same yield as reported by Michot in that experiment.

Michot uses distilled ClSI in example 10 but is silent about any residual content of compound of formula (3), that is CSI, in this distilled ClSI starting material. Comparative example (i) was also done with ClSI starting material, that was prepared by distillation under vacuum. The content of CSI in this ClSI starting material was determined to be 0.3 wt-%.

Since the same yield of 55% was obtained in our Comparative Example (i) as reported in D1, it is to be assumed that the residual content of ClSI was the same in the experiment of Michot.

### Example 8

The Comparative Example (i) was repeated with the difference that 1 g of MIXTURE-TRIPLE-85-7.5-7.5 was used instead of the reported 1 g ClSI.

The yield was 82%, which is considerably higher than the yield of 55% obtained with ClSI in the Comparative Example (i).

### Examples 9 to 15

Example 5 was repeated with the differences as stated in Table 1:

Under these conditions HFSI was produced.

TABLE 1

| | PRESSURE1-1 | TEMP1-1 | Yield |
|---|---|---|---|
| Example | [bar] | [° C.] | [%] |
| 9 | 80 | 140 | 88 |
| 10 | 80 | 110 | 58 |
| 11 | 60 | 160 | 81 |
| 12 | 40 | 160 | 84 |
| 13 | 20 | 160 | 84 |
| 14 | 12.5 | 160 | 74 |
| 15 | 5 | 160 | 72 |

### Examples 16 to 20

HFSI, prepared according to example 13, was diluted with water to provide a solution of HFSI in water with a concentration of HFSI of 14% by weight, the % by weight being based on the total weight of water and HFSI.

40 g of this solution of HFSI in water was mixed for 10 min with 20 g of solvent A. Two layers formed and were separated and the content of HFSI in the organic layer was determined by ¹⁹F NMR, providing the yield, details are given in Table 2.

TABLE 2

| | | | Yield of |
|---|---|---|---|
| | | HFSI in Organic Layer | Extraction |
| Example | Solvent A | [% by weight] | [%] |
| 16 | THF | 14.46 | 77.4 |
| 17 | ethylacetate | 15.22 | 79.6 |
| 18 | diethylether | 17.16 | 81.21 |
| 19 | MeTHF | 17.06 | 87.2 |
| 20 | 3,3-dimethyl-2-butanone | 16.91 | 63.5 |

### Examples 21 to 24

HFSI, prepared according to example 3, was diluted with water to provide a solution of HFSI in water with a concentration of HFSI of 5% by weight, the % by weight being based on the total weight of water and HFSI.

60 g of this solution of HFSI in water was mixed for 10 min with 20 g of solvent B. Two layers formed and were separated and the content of HFSI in the organic layer was determined by ¹⁹F NMR, providing the yield, details are given in Table 3.

TABLE 3

| | | | Yield of |
|---|---|---|---|
| | | HFSI in Organic Layer | Extraction |
| Example | Solvent B | [% by weight] | [%] |
| 21 | valeronitrile | 6.79 | 46.5 |
| 22 | MTBE | 10.46 | 84.8 |
| 23 | tert-butylacetate | 5.94 | 39.2 |
| 24 | diisopropylether | 5.45 | 38.1 |

### Example 25

The examples were carried out with
DEVICE1-0-HF: piston pump 260D from ISCO Teledyne
DEVICE1-0-COMP-II: piston pump 260D from ISCO Teledyne
DEVICE1-1 being a ¼ inch coiled tube with internal volume VOLUME1 of 32 ml made of hastelloy C. For the heating a coiled-tube-in-container set up was used. Heating medium was conventional oil.
DEVICE1-2 being a ⅛ inch tube with ca. 1.5 mL internal volume made of hastelloy C. Cooling was done by simply contact of the tube with the air having room temperature.
DEVICE1-3: pneumatically controlled valve from SAMSON Microvalve type 3510-7 with a Cv value of 0.01.
DEVICE1-4: any gaseous components, which are essentially HCl and excess HF, were separated from the reaction mixture in a vented vessel made of teflon.

MIXTURE-TRIPLE-85-7.5-7.5 was fed simultaneously with HF into DEVICE1-1 at a PRESSURE1-1 of 50 bar, MIXTURE-TRIPLE-85-7.5-7.5 was fed by DEVICE1-0-COMP-II at a flow rate of 4.6 ml/min, and HF was fed by DEVICE1-0-HF with at a flow rate of 1.77 ml/min. TIME1-1 was approximately 5 min, TEMP1-1 was 160° C. The molar ratio of HF:MIXTURE-TRIPLE-85-7.5-7.5 resulting from the flow rates was approximately 2.4:1. Then the reaction mixture from DEVICE1-1 was cooled to TEMP1-2 in DEVICE1-2, TEMP1-2 was room temperature, TIME1-2 was approximately 15 sec, and was then expanded by DEVICE1-3 into DEVICE1-4. The liquid collected was HFSI confirmed by ¹⁹F NMR.

The yield was 89% based on compound of formula (2) in MIXTURE-TRIPLE-85-7.5-7.5.

### Example 26—Mg(FSI)₂

HFSI is prepared according to example 11, and is diluted with water to provide a solution of HFSI in water with a concentration of HFSI of ca. 10.5% by weight, the % by weight being based on the total weight of water and HFSI. 426 g of this solution of HFSI in water is charged into a PTFE reactor and 130 g of methyl-tert butyl ether (MTBE) were added, mixed at 25° C. and the layers separated. The extraction was repeated. The organic layers were combined, they contained 309.3 g of HFSI (around 13% in MTBE, yield determined by ¹⁹F NMR).

103 g of the combined organic layers were charged back to the reactor and 13.5 g of Mg(OH)₂ suspension (20 wt-% in water) were added at a temperature between 10 and 20° C. After heating to room temperature most of the solid Mg(OH)₂ was dissolved and the organic layers was isolated. The MTBE was removed under vacuum at 60° C. until a concentration of around 40 wt-% Mg(FSI)₂ in a mixture of MTBE and water was obtained. Mg(FSI)₂ was crystallized under reduced pressure (200 to 5 mbar) and at a temperature of from 12 to 55° C.

133 g of 1,2-dichlorobenzene were dosed and simultaneously during this dosage MTBE and water were removed by distillation while keeping the level in the reactor constant. The conetn of the reactor was cooled to room temperature and a solid solidified in form of a slurry. After filtration of the slurry the filter cake was washed three times with 30 g of dichloromethane. After drying at 60° C. for three hours, white 5.8 g of Mg(FSI)₂ were isolated.

Mp=40 to 42° C.

### Example 27—1-Methyl-1-propylpyrolidinium FSI

HFSI is prepared according to example 11, and is diluted with water to provide a solution of HFSI in water with a concentration of HFSI of ca. 10.5% by weight, the % by weight being based on the total weight of water and HFSI.

426 g of this solution of HFSI in water is charged into a PTFE reactor and 130 g of methyl-tert butyl ether (MTBE) were added, mixed at 25° C. and the layers separated. The extraction was repeated. The organic layers were combined, they contained 309.3 g of HFSI (around 13% in MTBE, yield determined by ¹⁹F NMR).

102 g of the combined organic layers were charged back to the reactor and 12.6 g 1-methyl-1-propylpyrolidin.HCl were added at a temperature between 10 and 20° C. After heating to room temperature two layers had formed, the water layer was removed. To the organic layer 20 g of water were added and mixed for 15 min at room temperature. The layers were separated and the extraction repeated. The organic layer was dried with sodium sulfate, filtered and the MTBE was evaporated under reduced pressure of 17 mbar at 60° C. 14.6 g 1-methyl-1-propylpyrolidinium FSI were obtained as yellow liquid (the structure was confirmed with ¹H and ¹⁹F NMR).

### Example 28—NaFSI (from dimethylpentanone)

HFSI is prepared according to example 11, and is diluted with water to provide a solution of HFSI in water with a concentration of HFSI of ca. 10.5% by weight, the % by weight being based on the total weight of water and HFSI.

800 g of this solution of HFSI in water is charged into a PTFE reactor and mixed with 200 g of 2,4-dimethyl-3-pentanone at 25° C., two layers separated, The organic phase was separated. The extraction with 200 g of 2,4-dimethyl-3-pentanone was repeated. The combined organic layers (473.6 g of a solution of HFSI in 2,4-dimethyl-3-pentanone, yield determined by ¹⁹F NMR) were charged back to the reactor and 48.6 g of an aqueous NaOH solution (25 wt-%) were added at a temperature between 10 and 20° C. Two layers formed, the aqueous layer was discarded. The extraction with NaOH was repeated. 2,4-Dimethyl-3-pentanone was removed under vacuum at 60° C. until a solution with a concentration of around 30 wt-% of NaFSI was achieved. This solution was filtered through a 1 micrometer filter to remove respective small and solid particles. NaFSI was crystallized under reduced pressure (200 to 5 mbar) and at a temperature from 20 to 40° C. by dosing 103 g of 1,2-dichlorobenzene and simultaneous removal of 2,4-dimethyl-3-pentanone and water by distillation while keeping the level in the reactor constant. NaFSI crystallized in form of a slurry, after filtration of the slurry and washing of the filter cake three times with 100 g dichloromethane in total, 41.4 g of a filter cake of NaFSI were isolated. After drying of the filter cake at 60° C. for three hours, NaFSI was isolated as white powder.

### Example 29—Distillation of HFSI

HFSI, prepared according to example 25 (comprising 2.4 wt % or CSI and 77.07 wt % of HFSI) was distilled in a 1 liter batch column with 1 meter of Sulzer DX packing. The jacket temperature of the reboiler had a maximum of 140° C.

Fraction 1 to 6 were distilled at 60 mbar absolute pressure and a reflux of 8 to 1, fraction 7 and 8 were distilled at 23 mbar and a reflux of 4 to 1. Table 4 shows the results.

TABLE 4

| | Amount | CSI | HFSI |
|---|---|---|---|
| | [g] | [wt %] | [wt %] |
| Feed | 914.14 | 2.4 | 77.07 |
| Fraction 1 | 114.14 | 9.7 | 69.2 |
| Fraction 2 | 103.88 | 1.6 | 82.7 |
| Fraction 3 | 115.38 | 0.6 | 88.2 |
| Fraction 4 | 114.29 | 0.6 | 93.1 |
| Fraction 5 | 69.52 | 0 | 99.7 |
| Fraction 6 | 60.09 | 0 | 92.2 |
| Fraction 7 | 12 | 0 | ca. 100 |
| Fraction 8 | 12.16 | 0 | ca. 100 |
| Bottom | 104.95 | 0 | 51.01 |

With this distillation a purity of HFSI of over 99% can be achieved.

### Example 30—LiFSI Using LiCl (with Distilled HFSI)

10.78 g LiCl were suspended in 90.73 g dimethylcarbonate and cooled to below 10° C. To this mixture 50.26 g HFSI, prepared according to Example 29 by mixing Fractions 5 and 6, were dosed during 1.5 h while keeping the temperature in the range of from 0° C. to 10° C. After heating to 20 to 25° C. the solvent was removed by distillation under reduced pressure (40 to 50 mbar) and at 40° C. 15.84 g of distillate were obtained. The residue containing LiFSI was filtered using a 1 micron filter to remove remaining LiCl and small particles.

LiFSI was crystallized under reduced pressure (200 to 5 mbar) and at a temperature of from 20 to 60° C. by dosing 221 g of 1,2-dichlorobenzene and simultaneous removal of dimethylcarbonate by distillation while keeping the level in the reactor constant. LiFSI crystallized in form of a slurry, after filtration of the slurry and washing of the filter cake (three times with 150 g dichloromethane in total), 30.86 g of a filter cake of LiFSI were obtained. After drying of the filter cake at 60° C. for 3 h, 26.3 g of LiFSI was obtained as white powder.

### Example 31—LiFSI Using LiOH Monohydrate (with Distilled HFSI)

10.88 g LiOH monohydrate were suspended in 90.65 g diethylcarbonate and cooled to 0 to 10° C. To this suspension 50.11 g HFSI, prepared according to Example 29 by mixing Fractions 5 and 6, were dosed during 1.5 h while keeping the temperature in the range of from 0° C. to 10° C. After heating to 20 to 25° C. diethylcarbonate and water were removed by distillation under reduced pressure (50 to 60 mbar) and at 50° C. 6.82 g of distillate were obtained. The residue containing LiFSI was filtered using a 1 micron filter to remove remaining unsolved LiOH and small particles.

LiFSI was crystallized under reduced pressure (200 to 5 mbar) and at a temperature of from 20 to 60° C. by dosing 1,2-dichlorobenzene and simultaneous removal of dimethylcarbonate by distillation while keeping the level in the reactor constant. LiFSI crystallized in form of a slurry, after filtration of the slurry and washing of the filter cake (three times with 150 g dichloromethane in total), 27.03 g of a filter cake of LiFSI were obtained. After drying of the filter cake at 60° C. for 3 h, 23.06 g of LiFSI was obtained as white powder.

1. A method for preparation of a compound of formula (I) 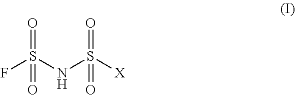
wherein the method comprises a step STEP1, a step STEPMIX and a step STEPEXTR;
wherein STEP1 comprises a reaction REAC1-1;
wherein in REAC1-1 a compound of formula (II) is reacted with HF 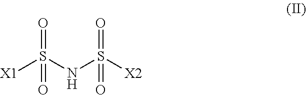
at a temperature TEMP1-1, wherein TEMP1-1 is at least 80° C.;
wherein at the beginning of REAC1-1, a compound of formula (III) is present in the reaction mixture; 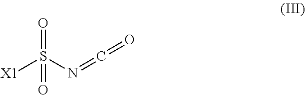
wherein the amount of the compound of formula (III), that is present in the reaction mixture at the beginning of REAC1-1, is at least 0.5%, wherein the % are % by weight and are based on the weight of the reaction mixture at the beginning of REAC1-1 excluding from said weight of the reaction mixture the weight of the HF;
wherein X is identical with X1 or with X2;
wherein X1 and X2 are identical or different and independently from each other selected from the group consisting of F, Cl, Br, I, RESF, and tolyl;
with the proviso that at least one of the residues X1 and X2 is Cl, Br, or I;
wherein RESF is fluorinated C₁₋₉ alkyl, which is unsubstituted or substituted by a substituent OCF₃;
wherein in STEPMIX, the compound of formula (I) is mixed with water by a mixing MIX, wherein MIX provides a mixture MIXWAT, and MIXWAT is the mixture of the compound of formula (I) with water,
wherein in STEPEXTR the compound of formula (I) is extracted from MIXWAT by an extraction EXTR, wherein EXTR is the extraction of the compound of formula (I) from MIXWAT with an organic solvent SOLVORG, and SOLVORG is an organic solvent that forms a biphasic system with water;
wherein EXTR provides the compound of formula (I) in a form of a solution SOLCOMP1,
wherein SOLCOMP1 is the solution of the compound of formula (I) in SOLVORG.

2. A method for preparation of a compound of formula (V); 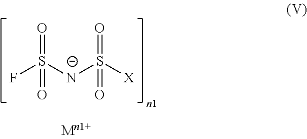
wherein n1 is 1, 2 or 3;
wherein M is selected from the group consisting of alkaline metal, alkaline earth metal and Al;
wherein the method comprises STEP1 and a step STEP2;
wherein STEP1 is as defined in claim 1;
wherein in STEP2 the H of the compound of formula (I) is exchanged against M.

3. The method according to claim 2, wherein
M is selected from the group consisting of Na, K, Li, Mg, and Al.

4. The method according to claim 2, wherein
the method further comprises STEPMIX and STEPEXTR;
wherein STEPMIX and STEPEXTR are as defined in claim 1.

5. A method for preparation of a compound of formula (I-AMI); 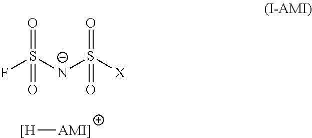
wherein AMI is a compound selected from the group consisting of N(R100)(R200)R300 and N(R400)R500;
R100, R200, R300 are identical or different and are selected from the group consisting of H, C₁₋₆ alkyl and halogenated C₁₋₆ alkyl; or
R100 and R200 form together with the N a saturated 5, 6, 7 or 8 membered heterocyclic ring RINGA;
R400 and R500 form together with the N an unsaturated 5, 6, 7 or 8 membered heterocyclic ring RINGB;
RINGA and RINGB can have 1 or 2 additional endocyclic heteroatoms selected from the group consisting of N, O and S;
RINGA and RINGB are unsubstituted or substituted by 1, 2 or 3 identical or different substituents selected from the group consisting of C₁₋₆ alkyl, halogenated C₁₋₆ alkyl, C₁₋₆ alkoxy and halogen;
wherein the method comprises STEP1 and a step STEP2-1;
wherein in STEP2-1 the H of the compound of formula (I) is exchanged against H-AMI; and
wherein STEP1 is as defined in claim 1.

6. The method according to claim 5, wherein
the method further comprises STEPMIX and STEPEXTR;
wherein STEPMIX and STEPEXTR are as defined in claim 1.

7. A method for preparation of a compound of formula (V); 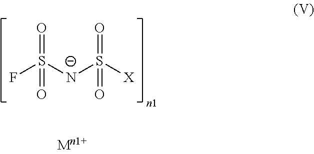
wherein n1 is 1, 2 or 3;
wherein M is selected from the group consisting of alkaline metal, alkaline earth metal and Al; and
wherein
the method comprises preparing the compound of formula (I-AMI) according to the method of claim 5 and a step STEP2-2;
wherein in STEP2-2 the H-AMI of the compound of formula (I-AMI) is exchanged against M to form the compound of formula (V).

8. The method according to claim 7, wherein
the method further comprises STEPMIX and STEPEXTR;
wherein STEPMIX and STEPEXTR are as defined in claim 1.

9. The method according to claim 1, wherein
STEP1 further comprises a purification PUR1, wherein in PUR1, the compound of formula (I) is purified by extraction, distillation, evaporation, membrane assisted separation, or a combination thereof.

10. (canceled)

11. (canceled)

12. The method according to claim 1, wherein
TEMP1-1 is at least 90° C.

13. The method according to claim 1, wherein
at least one of the residues X1 and X2 is Cl or Br.

14. The method according to claim 1, wherein
the compound of formula (II) is used for REAC1-1 in a form of a mixture MIX-II-III;
wherein MIX-II-III is a mixture of the compound of formula (II) with the compound of formula (III).

15. The method according to claim 1, wherein
the compound of formula (II) is prepared in a step STEP0;
wherein STEP0 comprises a reaction REAC0-1;
wherein REAC0-1 is a reaction of the compound of formula (III) with a compound of formula (IV); 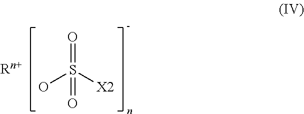
wherein X2 is as defined in claim 1;
Rⁿ⁺ is selected from the group consisting of H⁺, Li⁺, Na⁺, K⁺, Mg²⁺, Ca²⁺, Zn²⁺, Cu²⁺, Al³⁺, Ti³⁺, Fe²⁺, Fe³⁺, B²⁺, 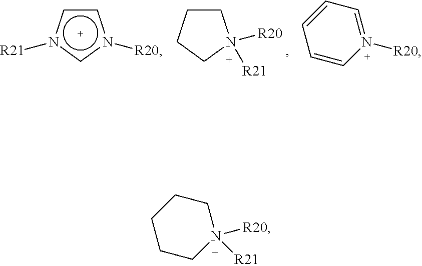
[N(R20)(R21)(R22)R23]⁺, and [P(R20)(R21)(R22)R23]⁺;
R20, R21, R22 and R23 are identical or different and independently from each other selected from the group consisting of H, C₁₋₈ alkyl, C₅₋₆ cycloalkyl, phenyl, benzyl, vinyl and allyl; and
N is 1, 2 or 3.

16. The method according to claim 3, wherein
the method further comprises STEPMIX and STEPEXTR;
wherein STEPMIX and STEPEXTR are as defined in claim 1.
